(19) United States
(12) Patent Application Publication
(10) Pub. No.: US 2024/0271096 A1
(43) Pub. Date: Aug. 15, 2024

(54) EFFICIENT DIFFERENTIATION OF SECOND HEART FIELD CARDIAC PROGENITOR CELLS AND RIGHT VENTRICLE CARDIOMYOCYTES FROM HUMAN PLURIPOTENT STEM CELLS

(71) Applicant: Wisconsin Alumni Research Foundation, Madison, WI (US)

(72) Inventors: Timothy Kamp, Madison, WI (US); Yukihiro Saito, Okayama (JP)

(21) Appl. No.: 18/440,260

(22) Filed: Feb. 13, 2024

Related U.S. Application Data
(60) Provisional application No. 63/484,677, filed on Feb. 13, 2023.

(51) Int. Cl. C12N 5/077; A61K 35/34; A61P 9/00

(52) U.S. Cl. CPC C12N 5/0657; A61K 35/34; A61P 9/00; C12N 2501/155; C12N 2501/33; C12N 2501/415; C12N 2501/727; C12N 2503/00; C12N 2506/45

(57) ABSTRACT

The present disclosure provides a method of generating right ventricle (RV) cardiomyocytes from pluripotent stem cells, populations of second heart field (SHF) cardiac progenitor cells and right ventricle (RV) cardiomyocytes produced by the method, a method of using these cells to study RV dysfunction related diseases, and a method of treating a subject with cardiac disease comprising administering to the patient the SHF cardiac progenitor cells or the RV cardiomyocytes.

### CROSS-REFERENCE TO RELATED APPLICATIONS

This application claims priority to U.S. provisional application No. 63/484,677, filed Feb. 13, 2023, the disclosure of which is expressly incorporated by reference herein.

### STATEMENT REGARDING FEDERALLY SPONSORED RESEARCH

This invention was made with government support under HL148690, EB007534 and HL134764 awarded by the National Institutes of Health and under 1648035 awarded by the National Science Foundation. The government has certain rights in the invention.

### INCORPORATION BY REFERENCE OF SEQUENCE LISTING PROVIDED ELECTRONICALLY

This application contains a Sequence Listing submitted as an electronic text file named “22-1116-US_SequenceListing_ST26.xml” having a size of 40,039 bytes, and created on Feb. 10, 2024. The information contained in this electronic file is hereby incorporated by reference in its entirety.

### BACKGROUND OF THE INVENTION

The number of patients with heart disease continues to increase worldwide. Advances in diagnosis and treatment of heart disease have identified a significant subset of patients with primarily right ventricular pathology, such as Brugada syndrome, arrhythmogenic right ventricular cardiomyopathy, certain forms of congenital heart disease, and right heart failure secondary to pulmonary arterial hypertension. The pathophysiology of these right ventricular diseases is incompletely understood, and there are limited therapeutic options for them, in contrast to the range of therapies addressing diseases that primarily impact the left ventricle.

During heart development, the left ventricle (LV) and the right ventricle (RV) arise from distinct populations of cardiac progenitor cells that can be identified by expression of distinct transcription factors. Cardiac progenitor cells in the first heart field (FHF) are TBX5-positive and Nkx2.5 positive, for example, and these FHF progenitors form the linear heart tube and develop primarily into LV and atria. Cardiac progenitor cells in the second heart field (SHF) are TBX5-negative and Nkx2.5-positive, and SHF progenitors migrate into the outflow tract of the heart tube and grow primarily into RV as well as the inflow tract to contribute to the atria. Many prior studies have demonstrated that human pluripotent stem cells can differentiate into contracting cardiomyocytes, but the chamber-specific and heart field-specific identity of these cardiomyocytes is only beginning to be explored in recent studies. For example, Zhang et al. (2019, Cell Stem Cell 24:802-811 e5) generated NKX2-5^{TagRFP}TBX5^{Clover2} reporter human induced pluripotent stem cells (hiPSCs) to isolate NKX2-5⁺/TBX5⁺ FHF-like cells and NKX2-5⁺/TBX5⁻SHF-like cells. When cardiomyocytes derived from those marker-containing progenitor cells were examined, cardiomyocytes from SHF-like cells were understood to be atrial-like cells. Pezhouman et al. (2021, Cardiovasc Res. 118(3):828-843) also reported a method for inducing FHF-like cells and SHF-like cells separately using different CHIR 99021 concentrations and seeding densities from HES3-TBX5-TdTomato^{+/W}/NKX2-5^{GFP/W} double reporter human embryonic stem cells (hESCs). Most of the induced cardiomyocytes from FHF-like and SHF-like cells were determined to be atrial-like myocytes. Andersen et al. (2018, Nature Communications 9:314) identified CXCR4 as a marker for SHF-like cells derived from pluripotent stem cells. CXCR4⁺ cardiac progenitor cells derived from hiPSCs were sorted; however, the reference did not mention whether CXCR4⁺ cardiac progenitor cells give rise to ventricular myocytes or atrial myocytes. In addition, Zhang et al. (2019, Nature Communications 10:2238) induced SHF-like cells from hiPSCs using a fibroblast medium supplemented with beta fibroblast growth factor (bFGF), but the SHF-like cells were used to induce cardiac fibroblasts and not cardiomyocytes. The art has provided no established method for specifically inducing RV-like cardiomyocytes from human pluripotent stem cells (hPSCs).

Thus, there remains a need in this art for methods and reagents for specifically producing therapeutically useful quantities of right ventricle-specific cardiomyocytes

### SUMMARY OF THE INVENTION

This invention provides methods and reagents that specifically differentiate hPSCs to SHF cardiac progenitor cells and RV cardiomyocytes.

Described herein are isolated populations and cell cultures of RV cardiomyocytes that can be used for functional studies in RV dysfunction-related disease models. In addition, isolated populations and cell cultures of SHF cardiac progenitor cells and RV cardiomyocytes can be administered to a subject having a need thereof to replace the subject's damaged cardiomyocytes and provided herein are methods for achieving therapeutic objectives and outcomes using isolated populations and cell cultures of RV cardiomyocytes provided herein.

In one aspect, the disclosure provides methods for producing a population of right ventricular (RV)-like cardiomyocytes from human pluripotent stem cells (hPSCs) comprising: (a) culturing hPSCs in the presence of a glycogen synthase kinase 3 (Gsk3) inhibitor to obtain a first population of cells, (b) thereafter culturing the first population of cells without the Gsk-3 inhibitor, (c) culturing the first population of cells with a Wnt/β-catenin signaling pathway inhibitor to obtain a second population of cells comprising second heart field (SHF) cardiac progenitor cells, and (d) culturing the second population of cells comprising SHF cardiac progenitor cells without a Wnt/β-catenin signaling inhibitor to obtain a third population of cells comprising RV cardiomyocytes, wherein an insulin signaling activator is added anytime between step (a) and step (b).

Yet another aspect of the present disclosure provides alternative methods for producing a population of right ventricular (RV)-like cardiomyocytes from human pluripotent stem cells (PSCs) comprising: (a) culturing hPSCs in the presence of a glycogen synthase kinase 3 (Gsk3) inhibitor to obtain a first population of cells, (b) thereafter culturing the first population of cells without the Gsk-3 inhibitor, (c) culturing the first population of cells with a Wnt/β-catenin signaling pathway inhibitor to obtain a second population of cells comprising second heart field (SHF) cardiac progenitor cells, and (d) culturing the second population of cells comprising SHF cardiac progenitor cells without a Wnt/β-catenin signaling inhibitor to obtain a third population of cells comprising RV cardiomyocytes, wherein a bone morphogenic protein (BMP) signaling inhibitor is added anytime between step (a) and step (c).

In certain embodiments, the insulin signaling activator is present in culture media for 24 to 48 hours. In certain embodiments, the Gsk-3 inhibitor is CHIR 99021, CHIR 98014, BIO-acetoxime, BIO, LiCl, SB 216763, SB 415286, AR A014418, 1-Azakenpaullone, and Bis-7-indolylmaleimide, or a combination thereof. In certain embodiments, the insulin signaling activator is insulin, IGF-1, S597, or a combination thereof. In certain embodiments, the BMP signaling inhibitor is dorsomorphin, dorsomorphin homologue 1, LDN-193189, K02288, LDN-214117, ML347, LDN-212854 or a combination thereof. In certain embodiments, the Wnt/β-catenin signaling pathway inhibitor is XAV939, IWR-1, IWR-2, IWR-3, IWR-4, IWR-5, IWP-1, IWP-2, IWP-3, or IWP-4.

In certain embodiments, the SHF cardiac progenitor cells are between 70% to 99% of the second population of cells without cell separation or cell selection steps. Differential expression of these markers provides a means for enriching the population of SHF cells using, inter alia, cell separation or cell selection steps. In certain embodiments, the RV cardiomyocytes are between 60% to 80% of the third population of cells without cell separation or selection steps. Differential expression of these markers provides a means for enriching the population of SHF cells using, inter alia, cell separation or cell selection steps.

Yet another aspect of the present disclosure provides a population comprising SHF cardiac progenitor cells or RV cardiomyocytes produced by the method described herein.

In another aspect, the RV cardiomyocytes have distinct phenotypic properties compared to cardiomyocytes produced by the same method but without BMP signaling inhibition in step (i). The phenotypic properties that can be measured are sarcomere formation, cell size, spontaneous contraction rate, Ca²⁺ transient event duration, contraction speed, and relaxation speed.

Yet another aspect of the present disclosure provides methods for screening a compound, for example a compound that can have beneficial or therapeutic properties, using RV cardiomyocytes derived from hPSCs comprising: a) culturing the RV cardiomyocytes with the compound, b) culturing in parallel the RV cardiomyocytes without the compound, c) measuring a functional parameter of the RV cardiomyocytes in (a) and in (b), and (d) comparing the measured functional parameter of the RV cardiomyocytes in (a) with the measured functional parameter of the RV cardiomyocytes in (b). In certain embodiments, the hPSCs are derived from patients that have RV dysfunction-related diseases. Yet another aspect of the present disclosure provides a method of treating a subject with RV dysfunction related diseases comprising administering to the subject a pharmaceutical composition comprising the SHF cardiac progenitor cells or RV cardiomyocytes produced by the method described herein.

In certain embodiments, the diseases are Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, and Eisenmenger Syndrome. In certain embodiments differential effects of the compounds on the cells in (a) and (b) are useful in identifying clinically useful compounds that can have therapeutic benefits for individuals having RV dysfunction-related diseases.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The patent or patent application file contains at least one drawing in color. Copies of this patent or patent application publication with color drawings will be provided by the Office upon request and payment of the necessary fee.

The disclosure will be better understood and features, aspects, and advantages other than those set forth above will become apparent when consideration is given to the following detailed description thereof. Such detailed description refers to the following drawings.

FIG. 1A-1B show characterization of human pluripotent stem cells (hPSCs) generated using the methods disclosed herein. FIG. 1A shows immunostaining of undifferentiated cell markers expressed in hiPSCs. The hPSCs expressed OCT4, SSEA-4, TRA-1-60, and TRA-1-81. Scale bars are 100 μm. FIG. 1B shows hematoxylin eosin staining of teratoma tissue derived from hiPSCs. The scale bar is 400 μm.

FIG. 2A-2C show that insulin-containing medium on differentiation day 0 resulted in suppression of TBX5-tdTomato expression in HES3-TBX5-TdTomato^{+/W}NKX2-5^{eGFP/W} reporter human embryonic stem cell-derived cardiac progenitor cells and myocytes. FIG. 2A is a schematic diagram of the cardiac differentiation protocol disclosed herein. B-27 supplement containing insulin was used in place of B-27 supplement minus insulin on differentiation day 0. FIG. 2B shows NKX2-5-eGFP and TBX5-tdTomato expression on differentiation day 7 corresponding to the cardiac progenitor stage. TBX5-tdTomato expression was low with insulin treatment. NKX2-5-eGFP expression was low both with insulin and without insulin. The scale bars are 200 μm. FIG. 2C shows NKX2-5-eGFP and TBX5-tdTomato expression on differentiation day 14 corresponding to the cardiomyocyte stage. TBX5-tdTomato expression was low with insulin treatment. NKX2-5-eGFP expression was high both with insulin and without insulin. The scale bars are 1000 μm.

FIG. 3 shows that cell culture treatment with 3 μg/mL insulin on differentiation day 0 resulted in low TBX5-tdTomato expression in cardiomyocytes on day 13. TBX5-tdTomato expression decreased with increasing doses of insulin 0.1, 0.3, 1, and 3 μg/mL insulin. At 10 μg/mL insulin, very low eGFP and TBX5-tdTomator expression were detected. The scale bar is 400 μm.

FIG. 4A-4E show that addition of insulin to culture medium on day 0 suppressed expression of first heart field (FHF) markers and enhanced expression of second heart field (SHF) markers also in reporterless human induced pluripotent stem cell-derived cardiac progenitor cells and myocytes. FIG. 4A is a schematic diagram of the cardiac differentiation protocol disclosed herein. B-27 supplement was used in place of B-27 supplement minus insulin on differentiation day 0. FIG. 4B shows gene expression profiles evaluated with quantitative PCR on day 7 (n=3 in each). Data are shown as means±standard deviation. *P<0.05, **P<0.01.

FIG. 4C shows immunostaining of CXCR4 on day 6. CXCR4 expression was high with insulin and low without insulin. FIG. 4D shows immunostaining of NKX2-5 and TBX5 on day 10. TBX5 expression was low with insulin. NKX2-5 was high both with and without insulin.

FIG. 4E shows immunostaining of NKX2-5 and cardiac Troponin T (cTnT) on day 10. Expression of both were high both with insulin and without insulin. The scale bars are 200 μm.

FIG. 5A-5B show an insulin effect on TGF-3/SMAD signaling. FIG. 5A shows phosphorylation of SMAD1/5 evaluated by Western blotting. FIG. 5B shows phosphorylation of SMAD2/3 evaluated by Western blotting.

FIG. 6A-6G show that treatment with dorsomorphin on differentiation day 0 induced second heart field cells from human induced pluripotent stem cells. FIG. 6A is a schematic diagram of the cardiac differentiation protocol disclosed herein. Cells were treated with 1 μmol/L dorsomorphin on differentiation day 0. FIG. 6B shows that dorsomorphin downregulated expression of first heart field (FHF) markers and upregulated expression of second heart field (SHF) markers on day 7 (n=3 in each). Data are shown as means±standard deviation. *P<0.01, **P<0.001.

FIG. 6C shows immunostaining of ISL (a SHF marker) on day 6. ISL1 fluorescence was higher with DM treatment than with control. FIG. 6D shows immunostaining of CXCR4 on day 7. CXCR4 fluorescence was higher with DM treatment than with control. FIG. 6E shows immunostaining of NKX2-5 and TBX5 on day 10. FIG. 6F shows flow cytometry data of cardiac troponin T (cTnT) on day 12. TBX5 expression was low with DM treatment. FIG. 6G shows percentages of cTnT-positive cells (n=4 in each). Data are shown as means±standard deviation. The scale bars are 200 μm. DM: dorsomorphin.

FIG. 7A-7C show that treatment with different concentrations of dorsomorphin on differentiation day 0 induced second heart field (SHF) cells from human induced pluripotent stem cells. FIG. 7A shows that dorsomorphin downregulated the expression of first heart field (FHF) markers and upregulated the expression level of second heart field (SHF) markers on day 7 (n=1 in each).

FIG. 7B shows that dorsomorphin-treated cells expressed NKX2-5, but TBX5 expression was suppressed on differentiation day 10. The scale bar is 100 μm. FIG. 7C shows phase contrast images of purified cardiomyocytes on differentiation day 20. DM: dorsomorphin.

FIG. 8 shows a comparison of gene expression profiles between control and dorsomorphin-treated cells from differentiation day 0 to day 7.

FIG. 9A-9C show differences in gene expression profiles associated with fatty acid metabolism between control cardiomyocytes and dorsomorphin-treated cell-derived cardiomyocytes on day 20 (n=3 in each group). FIG. 9A shows scatterplots of expression profiles of 84 genes related to fatty acid metabolism. Red and green dots denote significantly upregulated and downregulated genes (p<0.05 and fold change >2 or <0.5), respectively. FIG. 9B shows a list of genes with more than 2-fold higher expression than control cardiomyocytes. FIG. 9C shows a list of genes whose expression is less than half that of control cardiomyocytes. Genes in bold are genes related to fatty acid metabolism that are commonly expressed in adult cardiomyocytes. DM: dorsomorphin.

FIG. 10A-10D show the effect of DMH1 on heart field and myocyte subtypes. FIG. 10A shows immunostaining of TBX5 and NKX2-5 on day 10. TBX5 expression decreased with increasing dose of DMH1. NKX2-5 expression was present in all conditions. FIG. 10B shows immunostaining of NKX2-5 and cardiac troponin T (cTnT) on day 10. Expression of both were present in all conditions. FIG. 10C shows immunostaining of CXCR4 on day 7. CXCR4 expression was higher with DMH1 treatment compared to control. FIG. 10D shows immunostaining of MLC2v and α-actinin on day 35. Expression of both were present. The scale bars are 200 μm.

FIG. 11A-11D show that addition of dorsomorphin on differentiation day 0 induced right ventricle myocytes from human induced pluripotent stem cells. FIG. 11A shows immunostaining of cardiac Troponin T (cTnT) and NKX2-5 in day 20 cardiomyocytes. Expression of both were present. TBX5 expression was lower with DM treatment compared to control. NKX2-5 expression was present in all conditions. The scale bars are 100 μm. FIG. 11B shows gene expression profiles evaluated with qPCR on day 20 (n=3 in each). Data are shown as means±standard deviation. *P<0.05, **P<0.01.

FIG. 11C shows immunostaining of NKX2-5/TBX5 in differentiation day 20 cardiomyocytes. FIG. 11D shows immunostaining of MLC2v and α-actinin on differentiation day 35. Expression of both were present in both DM and control. The scale bars are 100 μm. DM: dorsomorphin.

FIG. 12A-12D show right ventricle myocyte induction from commercially available human induced pluripotent stem cell line, 201B7. FIG. 12A shows immunostaining of CXCR4 and TBX5 on day 7. CXCR4 expression was higher and TBX5 expression was lower in DM compared to control. FIG. 12B shows immunostaining of NKX2-5 and TBX5 on differentiation day 11. TBX5 expression was lower in DM compared to control. NKX2-5 expression was present in DMSO (control) and DMH1 treatment.

FIG. 12C shows immunostaining of cardiac troponin T and NKX2-5 on day 11. Expression of both were present. FIG. 12D shows immunostaining of MLC2v (a ventricular marker protein) and α-actinin on day 34. Expression of both were present. The scale bar is 100 μm. DM: dorsomorphin. cTnT: cardiac troponin T.

FIG. 13A-13J show phenotypic properties of RV cardiomyocytes derived from hiPSCs. FIG. 13A shows spontaneous contraction frequency of day 46 cardiomyocytes (n=3 in each). Data are shown as means±standard deviation. FIG. 13B shows immunostaining of α-actinin (left) and transmission electron microscopic (TEM) images (right) of cardiomyocytes on differentiation day 50. FIG. 13C shows immunostaining of MLC2v and α-actinin on day 52. Expression of both were present. The scale bars are 100 μm.

FIG. 13D shows representative calcium transients recorded from control (upper) and dorsomorphin-treated cell-derived cardiomyocytes (bottom) on day 34. FIG. 13E is a bar diagram showing time to peak calcium transient. FIG. 13F shows time to 50% decay. Data are shown as means±standard deviation. FIG. 13G shows time to 80% decay (n=7 in each). Data are shown as means±standard deviation. FIG. 13H shows the speed of contraction of cardiomyocytes. FIG. 13I shows time to peak contraction (n=5 in each). Data are shown as means±standard deviation. FIG. 13J shows time from start of contraction to end of relaxation (n=5 in each). Data are shown as means±standard deviation. DM: dorsomorphin.

FIG. 14A-FIG. 14D show differences in expression of electrical conduction-related genes between LV-like CMs (control) and RV-like CMs (DM or DMH1-dorsomorphin or dorsomorphin homologue 1 treatment). FIG. 14A shows gene expression of SCN5A encoding voltage sodium channel 1.5 (Naᵥ1.5) and GJA1 encoding Connexin 43 evaluated with qPCR on day 20 (n=4 in each). Data are shown as means±standard deviation. FIG. 14B shows immunostaining of Connexin 43 on differentiation day 34 of control cells and DM-treated cells. Expression of Connexin 43 was lower in DM compared to control. FIG. 14C shows immunostaining of Connexin 43 and Nav1.5 on differentiation day 34 of control cells and DMH1-treated cells. Expression of Connexin 43 and Nav1.5 were lower in DMH1 compared to control.

FIG. 14D shows gene expression profiles of evaluated with qPCR on day 24 (n=3 in each) of control and DMH1-treated cells. Red boxes indicate significant increase in expression of DMH1-treated cells.

FIG. 15A-FIG. 15B show time-dependent treatment of insulin and dorsomorphin on induction of RV-like CMs. FIG. 15A shows low immunostaining of TBX5 on day 12 in cells treated with dorsomorphin on day 0 for 24 hours (DO-1), on day 1 for 48 hours (D1-D3), or on day 3 for 48 hours (D3-5); but not on day 5 for 48 hours (D5-D7). Expression of TBX5 was high in control. FIG. 15B shows low immunostaining of TBX5 in cells treated with insulin on day 1 for 48 hours (D1-3); but not on day 3 and day 5 for 48 hours (D3-5 and D5-7). Expression of TBX5 was high in control.

FIG. 16A-FIG. 16B show differences in cell size between LV-like CMs (control) and RV-like CMS (DM). FIG. 16A show fluorescent images of cardiomyocytes staining with cTnT on differentiation day 50. cTnT expression was present in both control and DM. FIG. 16B is a summary of the cell area of cardiomyocytes in FIG. 16A.

FIG. 17 shows RV injury by an aluminum rod cooled with liquid nitrogen (cryoinjury model). Blue staining indicates fibrosis in the RV area.

### DETAILED DESCRIPTION OF THE DISCLOSURE

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations were interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The term “contacting” includes the physical contact of at least one substance to another substance.

“Subject” refers to a warm-blooded animal such as a mammal, preferably an adult human, or a human child, afflicted with or having the potential to be afflicted with, one or more diseases and disorders described herein

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, or slowing progression of one or more symptoms of the disease or disorder.

The terms “express” or “expression” refer to transcription and/or translation of a nucleic acid coding sequence resulting in production of the encoded polypeptide.

As used herein, the term “pluripotent stem cells” (PSCs) means cells capable of differentiating into the three germ layer cells of the early embryo that further develop into all cells of the tissues of the adult body. PSCs suitable for the differentiation methods disclosed herein include, but are not limited to, human embryonic stem cells (hESCs), human induced pluripotent stem cells (hiPSCs), non-human primate embryonic stem cells (nhpESCs), non-human primate induced pluripotent stem cells (nhpiPSCs).

As used herein, the term “iPSCs” refers to cells that have been genetically reprogrammed to express genes that render the pluripotent characteristics of the higher potency cells, such as ESCs, as described herein. The cells can be obtained by reprogramming non-pluripotent (e.g., multipotent or somatic) cells. As used herein, “iPSC cell derivation” means reprogramming a somatic cell to become pluripotent. In certain embodiments, PSCs are iPSCs derived from human peripheral blood cells. In certain embodiments, PSCs are 201B7 human iPSC cell line. In certain embodiments, PSCs are human ESC cell line genetically expressing HES3-TBX5-TdTomato^{+/W}/NKX2-5^{eGFP/W} reporter protein.

Generation of RV Cardiomyocytes from PSCs

The present disclosure provides methods for differentiating hPSCs into SHF cardiac progenitors specifically to obtain RV cardiomyocytes; in these methods differentiation is facilitated using insulin. The protocol comprises (i) activating Wnt/β-catenin signaling with a Gsk-3 inhibitor (Gi) to obtain a first population of cells; (ii) thereafter optionally culturing the first population of cells without the Gsk-3 inhibitor for a period of time; (iii) inhibiting Wnt/β-catenin signaling with a Wnt/β-catenin signaling inhibitor (Wi) to obtain a second population of cells comprising SHF cardiac progenitor cells; and (iv) culturing the second population of cells comprising SHF cardiac progenitor cells without Wnt/β-catenin signaling inhibitor to obtain a third population of cells comprising RV cardiomyocytes, wherein an insulin signaling activator is added anytime between step (i) and step (ii). In some embodiments, the insulin signaling activator is present in culture media for 24 hours to 48 hours.

In addition, the present disclosure provides methods for differentiating hPSCs into SHF cardiac progenitors specifically to obtain RV cardiomyocytes by inhibiting BMP signaling. Briefly, the protocol comprises (i) activating Wnt/β-catenin signaling by culturing hPSCs with a Gsk-3 inhibitor (Gi) to obtain a first population of cells; (ii) thereafter optionally culturing the first population of cells without the Gsk-3 inhibitor for a period of time; (iii) inhibiting Wnt/β-catenin signaling with a Wnt/β-catenin signaling inhibitor (Wi) to obtain a second population of cells comprising SHF cardiac progenitor cells; and (iv) culturing the second population of cells comprising SHF cardiac progenitor cells without Wnt/β-catenin signaling inhibitor to obtain a third population of cells comprising RV cardiomyocytes, wherein a BMP signaling inhibitor is added anytime between step (i) and step (iii). In some embodiments, the BMP signaling inhibitor is present in culture media for 24 hours to 48 hours.

As used herein, the terms “first heart field” (FHF) and “second heart field” (SHF) will be understood by those having ordinary skill in the art to refer to cardiac progenitors that give rise to left ventricle (LV) and right ventricle (RV) cardiomyocytes respectively. FHF progenitors form the linear heart tube and grow primarily into LV and atria in vivo, while SHF progenitors migrate into the outflow tract of the heart tube and grow primarily into RV as well as the inflow tract to contribute to the atria. SHF cardiac progenitors produced by the methods described herein express one or more of the following differentiation markers: CXCR4, ISL1, TBX1, FGF10, and CCK (Andersen et al., 2018, Nat Commun. 9(1): 3140; Kang et al., 2009, Dev Biol. 334(2): 513-522; De Soysa et al., 2019, Nature 572(7767): 120-124; Rochais et al., 2014, Cardiovasc Res. 104(3): 432-442) at higher level than FHF cardiac progenitor cells. FHF cardiac progenitors produced by the methods described herein express one or more of the following differentiation markers: TBX5, HCN4, CORIN, and HAND1 at higher level than SHF cardiac progenitor cells.

As used herein, the term “RV cardiomyocytes” refers to cells that display genotypic and phenotypic characteristics indicative of cardiomyocytes that made up the right ventricle.

Provided herein is a population of cells comprising SHF cardiac progenitor cells produced by the method described above. A person having ordinary skill in the art would understand that the terms “RV-like cardiomyocytes”, and “LV-like cardiomyocytes” used in the prior art have the same meaning as the terms used in this disclosure. This is because the cells produced by the disclosed method have comparable but not exact cellular phenotype with that of native heart cells, given that the environment of the developing human heart cannot be fully recapitulated in vitro. It was known in the art that native SHF and FHF cardiac progenitor cells were challenging to isolate because these cells are only present in a short time window during embryonic development (Zhang et al., 2021, Circ. Res. 129(4): 474-487). The scarcity of these cells has also hindered understanding of their molecular markers and other characteristics. The SHF cardiac progenitor cells produced by the methods disclosed herein have similar molecular and phenotypic footprints of native SHF cells as data have shown here; however, it has been contemplated that the SHF cells provided herein are not identical to the native cells due to the differences in their in vitro environment compared to that of native cells during embryonic development.

The phenotypic characteristics of RV cardiomyocytes that can be measured using any method commonly known in the art include, but are not limited to, spontaneous contracting rate, sarcomere formation, calcium transient, action potential, contraction/relaxation speed, contractile force, glycolipid metabolism, and duration of the start of contraction to relaxation.

The genotypic characteristics of RV cardiomyocytes includes expression of RV biomarkers including, but are not limited to, CCK and IRX1. RV cardiomyocytes also express cardiomyocyte biomarkers including, but not limited to, cardiac troponin (cTnT), NKX2-5, TNNT2, and NPPA. RV cardiomyocytes lack expression of LV biomarkers including, but not limited to, TBX5, and HAND1. RV cardiomyocytes lack expression of atrial markers including, but not limited to NR2F2.

As used herein, the terms “signaling inhibitor” and “signaling activator” refer to compounds that block and activate, respectively, communication between different molecules of a specified cellular signaling pathway.

As used herein, the terms “low level” and “high level” refer to quantitative expression of certain genes and/or proteins compared to expression of a reference protein.

As used herein, the terms “-negative” and “-positive” refer to the absence and presence, respectively, of detectable immunofluorescence signal of the corresponding protein.

As used herein, the term “GiWi protocol” refers to a method for differentiating PSCs into cardiac progenitors to obtain cardiomyocytes as set forth in U.S. Pat. No. 8,951,798B2, U.S. Pat. No. 9,453,201B2, and U.S. Pat. No. 9,663,764B2, the disclosures of which are expressly incorporated by reference herein. Briefly, the GiWi protocol produces cardiomyocytes from PSCs and involves a) activating Wnt/β-catenin signaling with a Gsk-3 inhibitor (Gi) b) removing the Gsk-3 inhibitor and culturing the cells for a period of time, and c) inhibiting Wnt/β-catenin signaling with a Wnt/β-catenin signaling inhibitor (Wi) to obtain cardiac progenitors that can be further differentiated into cardiomyocytes and specifically FHF cells, SHF cells, and RV and LV cells.

The following paragraphs aim to provide sufficient description and examples of each step of the disclosed methods, not to be bound by theory or mechanism of activating or inhibiting signaling pathways involved in producing the claimed cells as part of the differentiation pathways, induced by treatment of the cells by the steps of the disclosed methods. A person of ordinary skill in the art would recognize that other alternative and equivalent way of achieving the same activation or inhibition of each signaling pathway would produce the same results and cells as in the disclosed methods.

### Activation of Wnt/β-Catenin Signaling

As used herein, Wnt/β-catenin signaling activation means to increase β-catenin expression levels or activity, TCF and LEF expression levels, and/or β-catenin/TCF/LEF induced transcriptional activity, by modulating pharmacologically or genetically the function of one or more proteins that participate in the Wnt/β-catenin signaling pathway. For example, interaction of β-catenin with Axin, a member of the β-catenin destruction complex, can be disrupted in PSCs by contacting them with the compound 5-(Furan-2-yl)-N-(3-(1H-imidazol-1-yl)propyl)-1,2-oxazole-3-carboxamide (“SKL2001”), which is commercially available, e.g., as catalog no. 681667 from EMD4 Biosciences. Disruption of Axin-β-catenin interaction allows β-catenin to escape degradation and thus increases the net level of β-catenin to drive β-catenin signaling, which activates the TCF/LEF regulated transcriptional program to promote mesendoderm formation. Effective concentrations of SKL2001 to activate Wnt/β-Catenin signaling range from about 10 μM to about 100 μM and more specifically about 20 μM, 30 μM, 40 μM, 50 μM, 60 μM, 70 μM, 80 M, 90 μM or another concentration of SKL2001 from about 10 μM to about 100 μM.

In certain embodiments, activation of Wnt/β-catenin signaling is achieved by inhibition of Gsk-3 phosphotransferase activity or Gsk-3 binding interactions with Gsk-3 inhibitors. Inhibition of Gsk-3 phosphorylation of β-catenin reduces tonic degradation of β-catenin, and thus, increases β-catenin expression and activity that drive differentiation of pluripotent stem cells to an endodermal/mesodermal lineage. Gsk3 inhibition can be achieved pharmacologically or genetically via methods commonly known in the art. These include, but are not limited to, providing small molecules that inhibit Gsk-3 phosphotransferase activity, RNA interference-mediated knockdown of Gsk-3, and overexpression of dominant negative forms of Gsk-3. Dominant negative forms of Gsk3 are known in the art as described, e.g., in Hagen et al., 2002, J Biol Chem, 277:23330-23335, which describes a Gsk-3 comprising a R96A mutation.

In certain embodiments, the Wnt/β-catenin signaling pathway is activated by inhibiting Gsk-3 in PSCs by contacting the PSCs with a small molecule that inhibits Gsk-3 phosphotransferase activity or Gsk-3 binding interactions. Small molecule Gsk-3 inhibitors include, but are not limited to, CHIR 99021, CHIR 98014, BIO-acetoxime, BIO, LiCl, SB 216763, SB 415286, AR A014418, 1-Azakenpaullone, Bis-7-indolylmaleimide, and any combinations thereof. In certain embodiments, CHIR 99021 is used to inhibit Gsk-3 in PSCs in the differentiation methods described herein. Effective concentrations of Gsk-3 inhibitor CHIR99021 range from about 5 μM to about 20 μM, e.g., about 6 μM, 8 μM, 10 μM, 12 μM, 14 μM, 16 μM, or another concentration of CHIR99021 from about 5 μM to about 20 μM.

Gsk-3 activity can also be inhibited by RNA interference knockdown of Gsk-3. For example, Gsk3 expression levels can be knocked-down using commercially available siRNAs against Gsk-3, e.g., SignalSilence® GSK-3α/β siRNA (catalog #6301 from Cell Signaling Technology®, Danvers, Mass.), or a retroviral vector with an inducible expression cassette for Gsk3, e.g., a commercially available Tet-inducible retroviral RNAi system from Clontech (Mountainview, Calif.) Catalog No. 630926, or a cumate-inducible system from Systems Biosciences, Inc. (Mountainview, Calif.), e.g., the SparQ® system, catalog no. QM200PA-2.

Alternatively, the Wnt/β-catenin signaling pathway is activated by overexpressing β-catenin itself, e.g., human β-catenin (GenBank Accession Nos: X87838 and CAA61107.1 for nucleotide and protein sequences, respectively). β-catenin overexpression is inducible β-catenin overexpression achieved using, e.g., any of the just-mentioned inducible expression systems. Alternatively, a constitutively active, stabilized isoform of β-catenin is used, which contains point mutations S33A, S37A, T41A, and S45A as described, e.g., in Baba et al., 2005, Immunity, 23(6):599-609.

### Activation of Insulin Signaling

As used herein, insulin signaling activation means to increase insulin receptor activity, or increase insulin's downstream signaling including but are not limited to PI3K-AKT signaling, and/or MAPK signaling, by modulating pharmacologically or genetically the function of one or more proteins that participate in these signaling pathways. In one embodiment, the concentration of insulin used to activate insulin receptors is 3 μg/mL. A suitable working concentration range for insulin is between 1 to 10 μg/mL depending on cell lines. Other insulin receptor activators include but are not limited to IGF-1 or insulin-mimetics such as S597.

### Inhibition of BMP Signaling

As used herein, BMP signaling inhibition means pharmacologically or genetically modulating one or more proteins that participate in BMP-mediated gene transcription. In certain embodiments, BMP signaling inhibition is achieved by inhibiting BMP receptor activation and phosphorylation of SMAD1/5 proteins with small molecules. These molecules include, but are not limited to: dorsomorphin, dorsomorphin homologue 1 (DMH1), insulin, LDN-193189, K02288, LDN-214117, ML347, and LDN-212854. A suitable working concentration range for dorsomorphin and DMH1 is between 0.1 to 1 μM depending on cell lines. In certain embodiments, dorsomorphin is used at a working concentration of 1 μM. In certain embodiments, DMH1 is used at a working concentration of 0.5 μM.

Inhibition of BMP signaling can also be achieved by RNA interference to decrease expression of one or more targets in the BMP signaling pathway. For example in some cases, RNA interference is against BMP receptor itself.

### Inhibition of Wnt/β-Catenin Signaling

As used herein, Wnt/β-catenin signaling inhibition means inhibition of TCF/LEF-β-catenin mediated gene transcription. Inhibition of Wnt/β-catenin pathway signaling can be achieved in a various ways including, but not limited to: contacting the cells with small molecule inhibitors, RNA interference of, or blocking antibodies against functional canonical Wnt ligands or Wnt pathway receptors (e.g., Frizzled and LRP5/6); providing small molecules that promote degradation of β-catenin and/or TCF/LEF; gene interference knockdown of β-catenin and/or TCF/LEF; overexpression of a dominant negative forms of β-catenin lacking the sequence for binding to TCF/LEF; overexpressing Axin2 (which increases β-catenin degradation); providing a small molecule inhibitor of a TCF/LEF and β-catenin interaction; and providing a small molecule inhibitor of a TCF/LEF-β-catenin and DNA promoter sequence interaction.

In some embodiments, inhibition of Wnt/β-catenin signaling is achieved by contacting the cells with one or more small molecule inhibitors of a Wnt ligand (e.g., a small molecule that inhibit secretion of the Wnt ligand) or inhibit Wnt ligands and their corresponding receptors interaction. Suitable small molecule inhibitors include, but are not limited to, N-(6-Methyl-2-benzothiazolyl)-2-[(3,4,6,7-tetrahydro-4-oxo-3-phenylthieno[3,2-d]pyrimidin-2-yl)thio]-acetamide (“IWP2”) available commercially, e.g., as Sigma catalog no. IO536; 2-(3,4,6,7-tetrahydro-3-(2-methoxyphenyl)-4-oxothieno[3,2-d]pyrimidin-2-ylthio)-N-(6-methylbenzo[d]thiazol-2-yl)acetamide (“IWP4”) available commercially, e.g., as catalog no. 04-00306 from Stemgent (San Diego, Calif.); 4-(1,3,3a,4,7,7a-Hexahydro-1,3-dioxo-4,7-methano-2H-isoindol-2-yl)-N-8-quinolinyl-Benzamide (“IWR-1”) available commercially, e.g., as Sigma catalog no. 10161; Benzoic acid, 2-phenoxy-, 2-[(5-methyl-2-furanyl)methylene]hydrazide (“PNU-74654”), e.g., Sigma catalog no. P0052; or a combination thereof.

Other Wnt/β-catenin signaling inhibitors promote degradation of β-catenin. These compounds, directly or indirectly, stabilize Axin, which is a member of the β-catenin destruction complex, and thus enhance degradation of β-catenin. Examples of Axin-stabilizing compounds include, but are not limited to, 3,5,7,8-Tetrahydro-2-[4-(trifluoromethyl)phenyl]-4H-thiopyrano [4,3-d]pyrimidin-4-one (“XAV939”), e.g., Sigma catalog no. X3004; 4-(1,3,3a,4,7,7a-Hexahydro-1,3-dioxo-4,7-methano-2H-isoindol-2-yl)-N-8-quinolinyl-Benzamide (“IWR-1”) available commercially, e.g., as Sigma catalog no. 10161. Other compounds, directly or indirectly, activate casein kinase 1α (CK1), which is a member of the β-catenin destruction complex, and thereby enhances degradation of β-catenin. Examples of CK1-stabilizing compounds include, but are not limited to, 6-(Dimethylamino)-2-[2-(2,5-dimethyl-1-phenyl-1H-pyrrol-3-yl)ethenyl]-1-methyl-4,4′-methylenebis[3-hydroxy-2-naphthalenecarboxylate] (2:1)-quinolinium (“Pyrvinium pamoate salt hydrate”), e.g., Sigma catalog no. P0027.

A suitable working concentration range for such small molecule inhibitors is from about 0.1 μM to about 100 μM, e.g., about 2 μM, 5 μM, 7 μM, 10 μM, 12 μM, 15 μM, 18 μM, 20 M, 40 μM, 60 μM, 80 μM, or 100 μM or another working concentration of one or more the foregoing small molecule inhibitors ranging from about 0.1 μM to about 100 μM. In one embodiment, IWP2 or IWP4 are used at a working concentration of about 5 μM. In other embodiments, the above-mentioned small molecule inhibitors are used at the corresponding target IC₅₀.

Inhibition of Wnt/β-catenin signaling can also be achieved by RNA interference to decrease the expression of one or more targets in the Wnt/β-catenin pathway. For example in some cases, RNA interference is against β-catenin itself. Short hairpin interfering RNAs (shRNAs) can be used to knock down β-catenin expression, at least one of the following shRNA sequences are used:

(SEQ ID NO: 1)
CCGGAGGTGCTATCTGTCTGCTCTACTCGAGTAGAGCAGACAGATAGCA
CCTTTTTT
or
(SEQ ID NO: 2)
CCGGGCTTGGAATGAGACTGCTGATCTCGAGATCAGCAGTCTCATTCCA
AGCTTTTT

Such shRNAs may be transfected as synthetic shRNAs into the cells by a number of conventional methods known in the art. Alternatively, shRNA sequences may be expressed from an expression vector, e.g., from a plasmid expression vector, a recombinant retrovirus, or a recombinant lentivirus.

Inhibition of Wnt/β-catenin signaling can also be achieved by genetically modifying the cells to comprise an inducible expression cassette for expression of an interfering RNA, e.g., an shRNA against β-catenin, as exemplified herein. The use of an inducible expression cassette allows temporal control of β-catenin knockdown. Such temporal control is well suited to the timing of Wnt/β-catenin signaling inhibition used in the differentiation methods described herein.

Alternatively, inhibition of Wnt/β-catenin signaling can be achieved by using an antibody that blocks activation of a Wnt ligand receptor. The antibody can bind to one or more Wnt ligand family members and inhibit binding of the one or more Wnt ligands to their receptors. Such antibodies are known in the art, as described as, e.g., an anti-Wnt-1 antibody in He et al. (2004), Neoplasia, 6(1):7-14. Additionally, the blocking antibody can target against a Wnt ligand receptor and block interaction of Wnt ligands with the receptor, as described, e.g., in Gurney et al., 2012, Proc. Natl. Acad. Sci. USA, 109:11717-11722.

### Culture Media

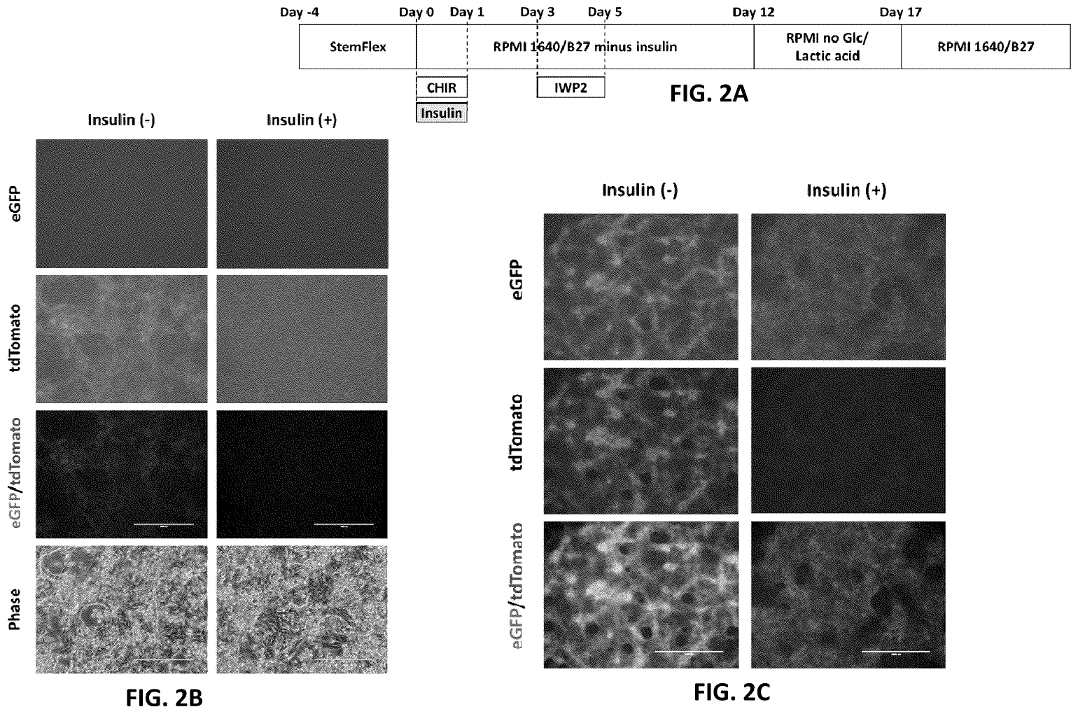

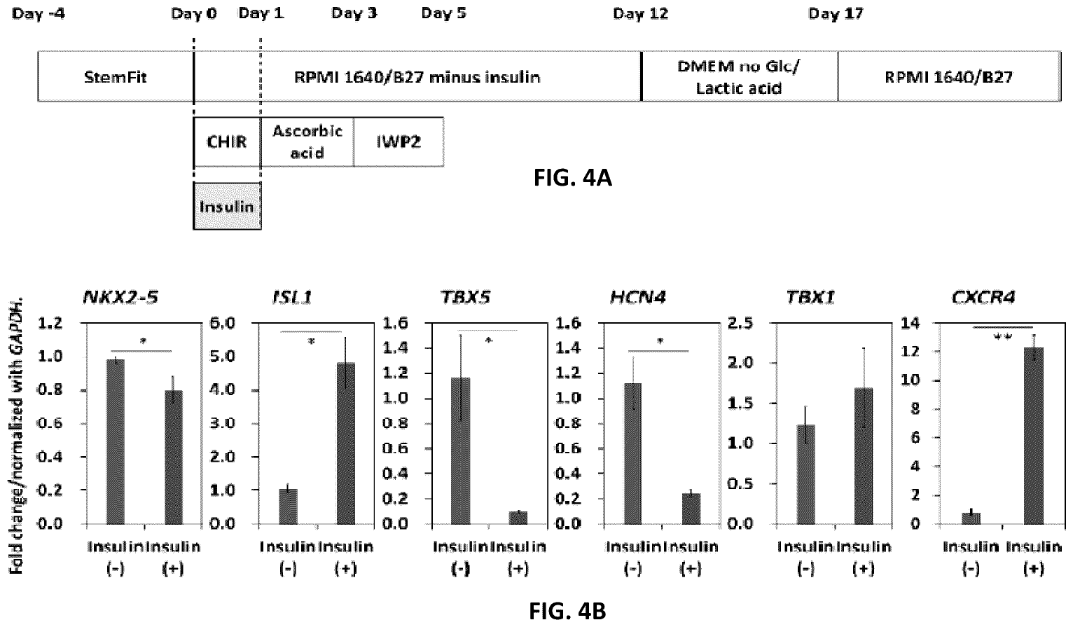

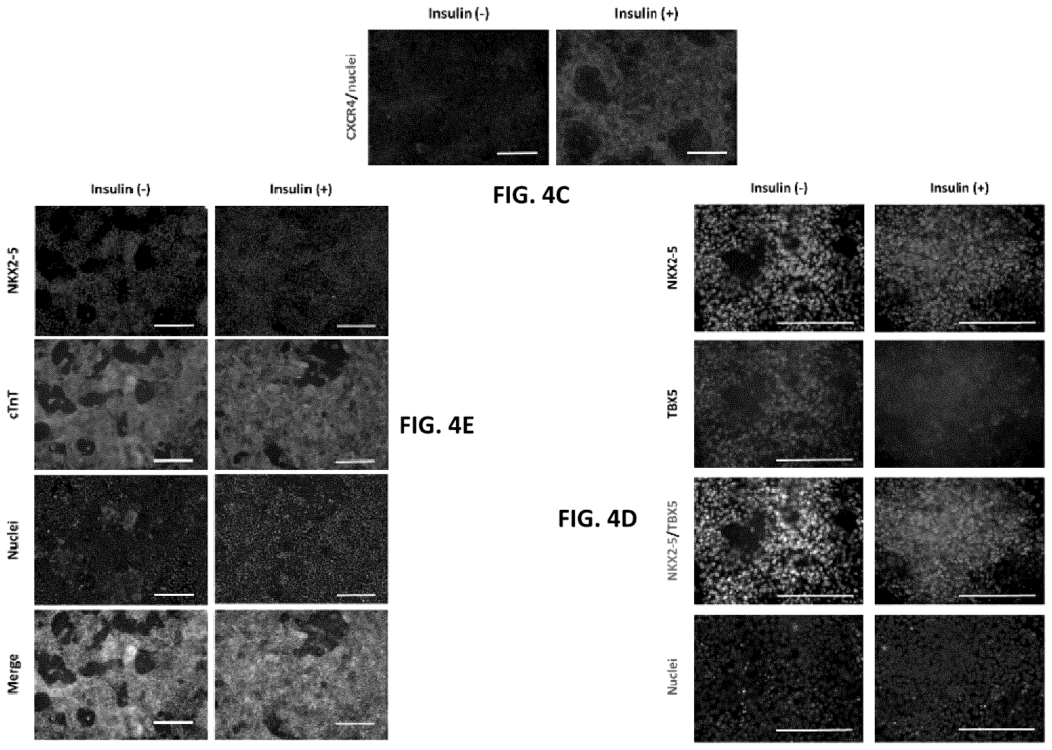

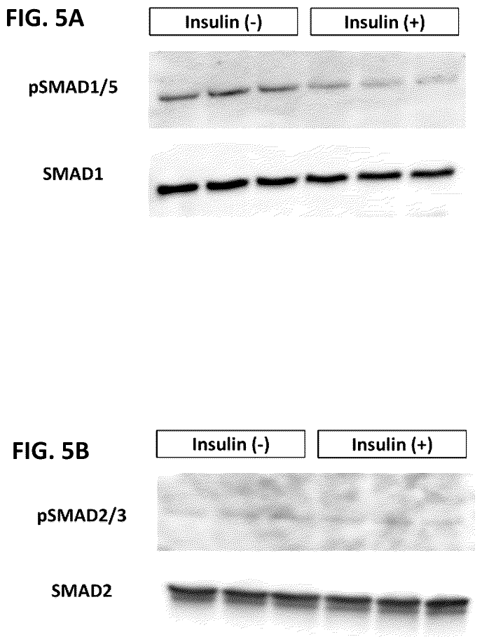

Defined media and substrate conditions for culturing pluripotent stem cells, as used in the methods described herein, are well known in the art. In some exemplary embodiments, PSCs to be differentiated according to the methods disclosed herein are cultured in StemFit AK02N (Ajinomoto, Tokyo, Japan), or StemFlex Medium (Life Technologies, Inc.) on a Matrigel substrate (Corning) or iMatrix-511 (Corning or Matrixome, Osaka, Japan) according to the manufacturer's protocol. For example, in embodiments comprising iMatrix-511, an amount of iMatrix-511 is diluted in phosphate-buffered saline (PBS) sufficient to coat a plate at 0.5 μg/cm², wherein the coated plates are allowed to stand at 37° C. for 1 hour. Schematic diagrams in FIG. 2A and FIG. 4A summarize the media used in the differentiation methods disclosed herein.

Throughout the differentiation methods provided herein, pluripotent cells are typically cultured in a medium substantially free of insulin, except when insulin is added to inhibit BMP signaling in the first step (i). In some embodiments, a medium comprising the RPMI 1640 Medium (Thermo Fisher Scientific) supplemented with B-27 (minus insulin) (Life Technologies, catalog no. 0050129SA) is used throughout the differentiation process. In some embodiments, B-27 Supplement (with insulin) is used when insulin is required.

A number of known base culture media are suitable for use throughout the differentiation methods described herein. Such cell base cell culture media include, but are not limited to, RPMI, DMEM/F12 (1:3), DMEM/F12 (1:1), DMEM/F12 (3:1), F12, DMEM, and MEM.

### Timing

In some embodiments, in the first step (i), PSCs are subjected to activation of Wnt/β-catenin signaling pathway for a period of about 24 hours to about 72 hours, e.g., about 24 hours, 36 hours, 48 hours, 60 hours, or 72 hours or another period of Wnt/β-catenin pathway signaling activation from about 24 hours to about 72 hours. In one embodiment, PSCs are subjected to Wnt/β-catenin signaling activation for a period of about 24 hours.

In some embodiments, in the second step (ii), after the end of the Wnt/β-catenin signaling activation step, i.e., after the agent for activating the Wnt/β-catenin signaling has been removed, the cells are cultured in the absence of external Wnt/β-catenin signaling activation for a period of at least about 0 hours to about 48 hours, e.g., about 0 hours, 24 hours, 48 hours or another period from at least about 0 hours to about 48 hours or another period from at least about 8 hours to about 60 hours. In one embodiment, this culture period is about 48 hours.

In some embodiments, in step (iii), immediately after the culturing period in the absence of Wnt/β-catenin signaling activation, the cells are subjected to inhibition of Wnt/β-catenin signaling pathway. In some embodiments, step (iii) is initiated at least about 48 hours to about 96 hours following the beginning of step (i), e.g., at least about 48 hours, 72 hours, 96 hours, or another time point from at least about 48 hours to about 96 hours following the beginning of step (i). In other embodiments, step (iii) begins 72 hours after the beginning of step (i).

Typically, inhibition of Wnt/β-catenin signaling in the first population of cells during step (iii) is maintained for a period of at least about 1 day to about 3 days, e.g., about 1 day, 2 days, 2.5 days, 3 days or another period of Wnt/β-catenin signaling inhibition from at least about 1 day to about 3 days. In some embodiments, where a small molecule inhibitor is used to inhibit Wnt/β-catenin signaling, the cells are contacted with the small molecule for a period of about 2 days, and then culture of the cells continues in the substantial absence of the small molecule inhibitor.

In some embodiments, activation of an insulin signaling pathway is initiated by addition of an insulin signaling activator in step (i) or step (ii), and lasts for the same period as step (i) or step (ii). In some embodiments, the insulin signaling activator is added at the same time as Wnt/β-catenin signaling activation is initiated. In some embodiments, the insulin signaling activator is added after Wnt/β-catenin signaling activation is ended, i.e., the start of step (ii). In some embodiments, the activation of insulin signaling pathway lasts for 24 hours to 48 hours.

In some embodiments, inhibition of BMP signaling pathway is initiated by addition of a BMP signaling inhibitor in step (i), step (ii), or step (iii) and lasts for the same period as step (i), step (ii), or step (iii). In some embodiments, the BMP signaling inhibitor is added at the same time as Wnt/β-catenin signaling activation is initiated. In some embodiments, the BMP signaling inhibitor is added after Wnt/β-catenin signaling activation is ended, i.e., the start of step (ii). In some embodiments, the BMP signaling inhibitor is added at the same time as Wnt/β-catenin signaling inhibition is initiated, i.e., the start of step (iii). In some embodiments, the inhibition of BMP signaling pathway lasts for 24 hours to 48 hours.

While cells are cultured continuously from the beginning of step (i) to step (iii) to obtain a population comprising SHF cardiac progenitors, in some cases, cultured cells can be removed from a culture substrate and frozen for storage thus permitting the differentiation method to be resumed after thawing cells at a later date. For example, in some cases, the first population of cells obtained after step (i) is collected and stored frozen in any number of suitable cell cryopreservation media known in the art, and then later thawed and cultured to resume the differentiation method starting at step (ii) and continuing to step (iii) in which Wnt//β-catenin pathway signaling is inhibited to drive differentiation into a second cell population comprising cardiomyocytes.

Alternatively, after step (iii), the cells obtained comprising SHF cardiac progenitors, are cryopreserved, and thawed at a later date for continued culture of the cells in order to obtain a population comprising RV cardiomyocytes. Accordingly, one of ordinary skill in the art will appreciate that, where the differentiation methods described herein include a cell freezing step, the absolute time interval between at least two steps will be different from the corresponding step interval in embodiments that do not include a freezing step.

Typically, after step (iii), the cells obtained by the disclosed methods comprises a very high proportion of SHF cardiac progenitors. In some embodiments, the SHF cardiac progenitors comprises about 70% to about 99% of total cells obtained after step (iii), e.g., about 70%, 75%, 80%, 85%, 90%, 95%, 98%, or another percent of SHF cardiac progenitors from about 70% to about 99% of total cells obtained after step (iii).

In some embodiments, after ending the inhibition of Wnt/β-catenin signaling initiated during step (iii), as described herein, the resulting population of cells, comprising SHF cardiac progenitors, is cultured for an additional period of time to obtain a cell population comprising RV cardiomyocytes. In some embodiments, the additional cell culture period ranges from at least about 5 days to about 200 days, e.g, about 5 days, 10 days, 15 days, 20 days, 25 days, 30 days, 35 days, 40 days, 45 days, 55 days, 70 days, 90 days, 100 days, 120 days, 150 days, 170 days, 180 days, 190 days, 200 days or another culture period, after ending inhibition of Wnt/β-catenin signaling, from at least about 5 days to about 200 days following the end of Wnt/β-catenin signaling inhibition. In one embodiment, the cells obtained from step (iii) comprising SHF cardiac progenitors is cultured for a period of at least about 5 days after ending inhibition of Wnt/β-catenin signaling.

In some embodiments, continued culture of the cells obtained from step (iii) comprising SHF cardiac progenitors (in the absence of Wnt/β-catenin signaling inhibition) yields a cell population comprising about 60% to about 80% RV cardiomyocytes, e.g., about 60%, 66%, 72%, 75%, 80%, or another percent of RV cardiomyocytes from about 60% to about 80%.

The above-mentioned RV cardiomyocytes, obtained after culturing of the cells obtained from step (iii) comprising SHF cardiac progenitors, can be subjected to an additional cell separation/cell selection step commonly known in the arts to obtain a population of cells with at least 70% cTnT-positive cells. In some embodiments, the cell selection step is a metabolic selection step where in glucose in the medium is replaced with lactic acid. Alternatively, cell separation or enrichment methods, e.g., FACS, MACS, or laser-targeted ablation of non-cardiomyocytes are used to obtain a population of cells enriched with cardiomyocytes relative to the population of cells obtained after step (iii) prior to application of a cell separation or enrichment method.

Method for screening compounds against RV-related dysfunction using RV cardiomyocytes

This invention provides methods for screening a compound, for example a compound that can have beneficial or therapeutic properties, using RV cardiomyocytes derived from hPSCs comprising: a) culturing the RV myocytes with the compound, b) culturing in parallel the RV cardiomyocytes without the compound, c) measuring a functional parameter of the RV cardiomyocytes in (a) and in (b), and d) comparing the measured functional parameter of the RV cardiomyocytes in (a) with the measured functional parameter of the RV myocytes in (b).

In some embodiments, the hPSCs are derived from patients that have RV dysfunction-related diseases. In some embodiments, the diseases include, but are not limited to, Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, and/or Eisenmenger Syndrome.

As used herein, the term “compound” refers to natural or synthetic molecules including for examples dorsomorphin, DMH1, and insulin.

The term “functional parameters” refers to parameters used to measure cardiac function of myocytes known in the art, including but not limited to, beating rate frequency, action potential duration, action potential shape, action potential conduction velocity, conduction pattern (reentry, rotors fibrillatory) (Campbell et al., 2012, Physiol, 590:6363-79; Hou et al., 2010, Circ Res. 107:1503-I I; Kleber et al., 2004, Physiol Rev. 84:431-88), intracellular calcium cycling, contraction, and metabolic fluxes. Any means known in the art for measuring or observing these functional parameters may be used. In some embodiments, these functional characteristics are measured by optical mapping. In some embodiments, intracellular calcium cycling is measured using fluorescent dyes including but not limited to Fura2 and Fluo4. In some embodiments, contraction is measured by video edge detection or matrix bead displacement. In some embodiments, metabolic flux and metabolic properties of the co-culture are measured using the Seaborse assay system.

Method of Treating Cardiac Disease with SHF Cardiac Progenitor Cells and RV Cardiomyocytes

This invention provides methods for treating a patient with cardiac disease comprising administering to the patient a pharmaceutical composition comprising SHF cardiac progenitor cells or RV cardiomyocytes produced by the methods provided herein.

It is expected that SHF cardiac progenitor cells derived from PSCs as described herein retain some regenerative properties that are therapeutic to injured cardiac tissues. Unlike other tissues, cardiac tissues are generally non-regenerative because cardiomyocytes have limited capacity to proliferate and differentiate.

In some embodiments, SHF cardiac progenitor cells or RV cardiomyocytes as disclosed herein can be formulated in a pharmaceutical composition comprising pharmaceutically acceptable carriers and delivered to the site of cardiac injury. In certain embodiments, such pharmaceutical compositions are suitable for administration to a human or non-human animal via any one or more routes of administration using methods known in the art. Delivery routes for SHF cardiac progenitor cells and RV cardiomyocytes include, but are not limited to, intravenous injection, intramyocardial injection, intracoronary injection, intrapericardial transplantation, catheter-based transendocardial delivery and epicardial patches. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations can also contain compatible solid or liquid fillers, diluents, or encapsulating substances, which are suitable for administration to a human. Other contemplated carriers, excipients, and/or additives, which can be utilized in the formulations described herein include, for example, antimicrobial agents, antioxidants, antistatic agents, lipids, protein excipients such as serum albumin, gelatin, casein, salt-forming counterions such as sodium, and the like. These and additional pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Materials and Methods

### Human Pluripotent Stem Cell Culture

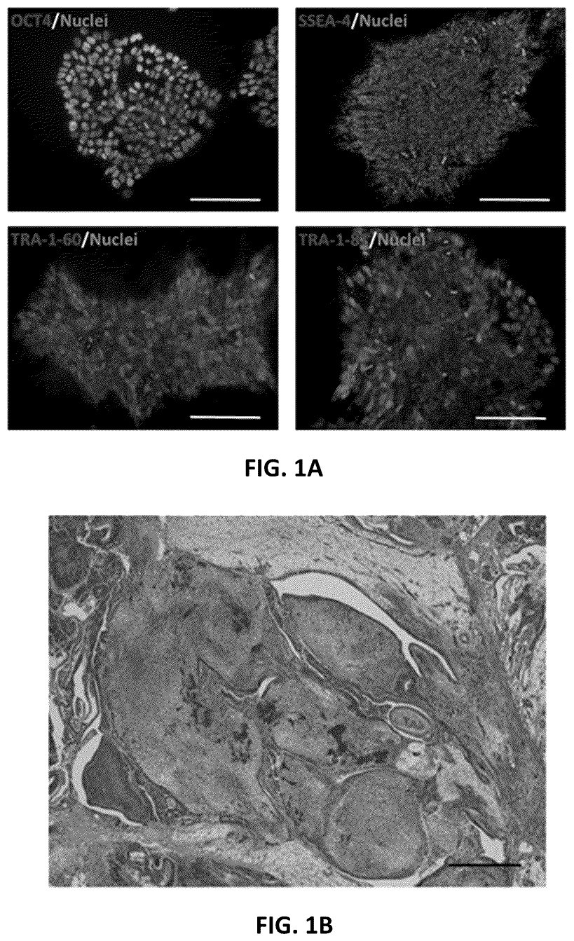

Human iPSCs (hiPSCs) were established from a healthy Japanese male donor (FIG. 1), 201B7 hiPSCs (RIKEN BRC, Tsukuba, Japan), and HES3-TBX5-TdTomato^{+/W}/NKX2-5^{eGFP/W}reporter hESCs that were a kind gift from Dr. Reza Ardehali, UCLA Geffen School of Medicine (Pezhouman et al., 2022, Cardiovasc Res. 118(3):828-43), were used. HiPSCs were maintained on iMatrix-511 (Matrixome, Osaka, Japan)-coated 6-well plates (Corning, Glendale, Arizona, United States) in StemFit AK02N (Ajinomoto, Tokyo, Japan) and passaged every 7 days.

HESCs were maintained on Matrigel Growth Factor Reduced Basement Membrane Matrix (Corning)-coated 6-well plates (Corning) in StemFlex Medium (Thermo Fisher Scientific, Waltham, MA, United States) and passaged every 3-4 days. StemPro Accutase Cell Dissociation Reagent (Thermo Fisher Scientific) was used for dissociation. 10 μmol/L Y27632 (Tocris, Bristol, United Kingdom) was used for improvement of cell survival for 24 hours after seeding. Mycoplasma tests were performed for all the cell culture in this study.

Generation of hiPSCs

Peripheral mononuclear cells (PMNCs) were collected from a 34-year-old healthy male according to the protocol published by Center for iPS Cell Research and Application, Kyoto University (Yamakawa et al., 2013, Stem Cells 31:458-66), purified with BD Vacutainer CPT (BD Biosciences, Franklin Lakes, NJ) according to the manufacturer's instructions, and a hiPSC line was generated as follows. First, 0.88 μg of pCXLE-hOCT3/4-shp53-F (#27077, Addgene, Watertown, MA, USA), 0.88 μg of pCXLE-hSK (#27078, Addgene), 0.88 μg pCXLE-hUL (#27080, Addgene) and 0.5 μg pCXWB-EBNA1 (#37624, Addgene) were electroporated into 3×10⁶ μMNCs with Nucleofector II (Lonza, Basel, Switzerland) and a Human T-cell Nucleofector Kit (Lonza) according to the manufacturer's instructions using program V-024. Thereafter, 3×10¹ cells were seeded onto six-well plates covered with an SNL 76/7 feeder layer (#07032801, European Collection of Authenticated Cell Cultures, UK). The transfected cells were cultured in X-vivo 10 Serum-free Hematopoietic Cell Medium (Lonza) supplemented with 10 ng/mL recombinant human Interleukin-2 (PeproTech Inc., Rocky Hill, NJ) and 6.7 l/well of Daybeds Human T-activator CD3/CD28 (Thermo Fisher Scientific). At 2 days, 4 days and 6 days after transfection, 1.5 mL of human ESC medium comprising 80% DMEM/F12 (Thermo Fisher Scientific) supplemented with 20% knockout serum replacement (Thermo Fisher Scientific), 100 mol/L 2-mercaptoenthanol (2-ME), MEM Non-Essential Amino Acids Solution (100×) (Thermo Fisher Scientific), and 10 ng/mL basic fibroblast growth factor (bFGF; Wako, Tsukuba, Japan) was added to each well without aspiration of the previous medium. The culture medium was then replaced with human ESC medium 8 days after transfection. Resulting colonies were picked 20 days after plating and expanded on iMatrix-511-coated plates with StemFit AK02N.

Differentiation of hPSCs to Cardiomyocytes

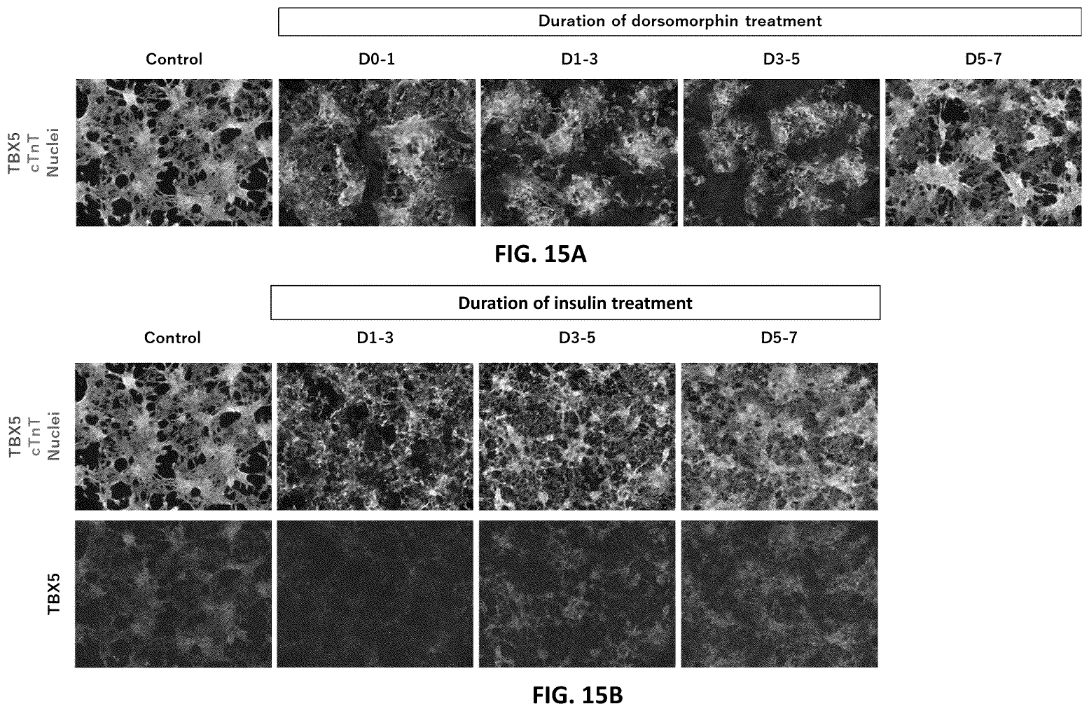

For LV-cardiomyocyte differentiation, cardiac differentiation was performed according to the protocol published by (Lian et al., 2012, Proc Natl Acad Sci USA 109:E1848-57). Cells were dissociated with StemPro Accutase Cell Dissociation Reagent at 37° C. for 5 minutes. HESCs were seeded on Matrigel-coated 24-well plates (Corning) in StemFlex Medium supplemented with 10 μmol/L Y-27632. HiPSCs were seeded on iMatrix-511-coated 12-well plates (Corning) in StemFit AK02N supplemented with 10 μmol/L Y-27632 at seeding densities of 1×10⁵ cells/cm² for hESCs and 1.25-2.5×10⁴ cells/cm² for hiPSCs. Cells were cultured for 4 days until the start of differentiation (day 0). On day 0, the medium was changed to RPMI 1640 Medium (Thermo Fisher Scientific) supplemented with B-27 Supplement minus insulin (Thermo Fisher Scientific) (RPMI/B27-insulin) or B-27 Supplement with insulin (Thermo Fisher Scientific), and 12 μmol/L CHIR 99021 (SelleckChem, Houston, TX, United States), and the cells incubated in this medium for 24 h (day 1). On day 1, the medium was changed to RPMI/B27-insulin. To enhance cardiac differentiation, 50 μmol/L 2-O-α-D-glucopyranosyl-L-ascorbic acid (Tokyo Chemical Industry, Tokyo, Japan) was added on day 1 only for hiPSCs (Burridge et al., 2014 Nat Methods, 11:855-860). On day 3, the medium was changed to RPMI/B27-insulin supplemented with 5 μmol/L IWP2 (SelleckChem) and on day 5, the medium was changed to 1 ml RPMI/B27-insulin. From day 7, the cells were cultured with RPMI/B27-insulin, and the cells were fed every other day. On day 12, the cells were washed with PBS (Thermo Fisher Scientific) and cultured with DMEM or RPMI, no glucose supplemented with 8 mmol/L L-(+)-Lactic acid (Merck) (Tohyama et al., 2013 Cell Stem Cell, 12:127-37). The medium was changed every other day until day 17. After day 17, the cells were cultured with RPMI/B27 and fed every 2 to 3 days. For long-term culture, the cells were dissociated with TrypLE™ Select Enzyme (10×) (Thermo Fisher Scientific) at 37° C. for 15-20 minutes and seeded on Matrigel- or iMatrix-511-coated plates. For RV-cardiomyocyte induction, Dorsomorphin 2HCl (SelleckChem), dorsomorphin homologue 1 (DMH1) (SelleckChem), or 3 μg/L insulin were used from day 0 to day 1. For FIG. 15A and FIG. 15B, dorsomorphin (DM) or 3 μg/L insulin were added on day 0 (for 24 hours), 1, 3, or 5 (for 48 hours).

Quantitative PCR (qPCR)

Cells were lysed with TRIzol™ Reagent (Thermo Fisher Scientific). Total RNA was extracted using a PureLink™ RNA Mini Kit (Thermo Fisher Scientific). Complementary DNA was synthesized using SuperScript™ IV VILO Master Mix with ezDNase™ Enzyme (Thermo Fisher Scientific) as prescribed by the manufacturer. PowerUp™ SYBR Green Master Mix (Thermo Fisher Scientific) and QuantStudio 1 (Thermo Fisher Scientific) were used for quantitative PCR (q-PCR), wherein q-PCR data were processed using the ΔΔCT method. These q-PCR experiments were performed in technical duplicate. PCR primers were purchased from Integrated DNA Technologies (Coralville, IA, USA) and details are shown in Table 1. An RT² Profiler PCR Array Human Fatty Acid Metabolism (Qiagen, Hilden, Germany) was used to examine fatty acid metabolism genes because it has been reported that there are differences in lipid metabolism between the LV and RV. Data were analyzed with the web-based tool of GeneGlobe Data Analysis Center (Qiagen).

TABLE 1
PCR primers.

| | SEQ | | | | Annealing |
|---|---|---|---|---|---|
| | ID | | | Product | temperature |
| Gene | NO | Orientation | Sequence | size (bp) | (° C.) |
| CCK | 3 | Forward | AAGCTCCTTCTGGACGAATGTC | 96 | 60 |
| | 4 | Reverse | AATCCATCCAGCCCATGTAGTC | | |
| CXCR4 | 5 | Forward | TCATCTCCAAGCTGTCACACTC | 170 | 60 |
| | 6 | Reverse | GTTCTCAAACTCACACCCTTGC | | |
| FGF10 | 7 | Forward | TTGAGAAGAACGGGAAGGTCAG | 144 | 60 |
| | 8 | Reverse | GTTTCCCCTTCTTGTTCATGGC | | |
| GAPDH | 9 | Forward | CAACGACCACTTTGTCAAGCTC | 144 | 60 |
| | 10 | Reverse | TCTCTTCCTCTTGTGCTCTTGC | | |
| HAND1 | 11 | Forward | TCAAGGCTGAACTCAAGAAGGC | 122 | 60 |
| | 12 | Reverse | GGTGCGTCCTTTAATCCTCTTC | | |
| HCN4 | 13 | Forward | GGTGTCCATCAACAACATGG | 66 | 60 |
| | 14 | Reverse | GCCTTGAAGAGCGCGTAG | | |
| MYL2 | 15 | Forward | GTGCTGAAGGCTGATTACGTTC | 121 | 60 |
| | 16 | Reverse | TGTAGTCCAAGTTGCCAGTCAC | | |
| ISL1 | 17 | Forward | TCTGAGGGTTTCTCCGGATTTG | 151 | 60 |
| | 18 | Reverse | GCATTTGATCCCGTACAACCTG | | |
| NKX2-5 | 19 | Forward | GTCCCCTGGATTTTGCATTCAC | 100 | 60 |
| | 20 | Reverse | ATAATCGCCGCCACAAACTCTC | | |
| NPPA | 21 | Forward | TCGATCTGCCCTCCTAAAAAGC | 139 | 60 |
| | 22 | Reverse | TCAGTACCGGAAGCTGTTACAG | | |
| NR2F2 | 23 | Forward | TCGCCTTTATGGACCACATACG | 149 | 60 |
| | 24 | Reverse | TTCCACATGGGCTACATCAGAG | | |
| SCN5A | 25 | Forward | AGAAGATGGTCCCAGAGCAATG | 131 | 60 |
| | 26 | Reverse | AATCTGCTTCAGAACCCAGGTC | | |
| TBX1 | 27 | Forward | GTGGATGAAGCAAATCGTGTCC | 197 | 60 |
| | 28 | Reverse | TGAATCGTGTCTCCTCGAACAC | | |
| TBX5 | 29 | Forward | TCATCAGTACCACTCTGTGCAC | 199 | 60 |
| | 30 | Reverse | GAGTGCAGATGTGAACATTGGG | | |
| TNNT2 | 31 | Forward | TTCACCAAAGATCTGCTCCTCGC | 166 | 60 |
| | | | T | | |
| | 32 | Reverse | TTATTACTGGTGTGGAGTGGGTG | | |
| | | | TGG | | |
| TGFB1 | 33 | Forward | ACAATTCCTGGCGATACCTCAG | 185 | 60 |
| | 34 | Reverse | GTTGATGTCCACTTGCAGTGTG | | |
| COL1A1 | 35 | Forward | GGTTTCAGTGGTTTGGATGGTG | 97 | 60 |
| | 36 | Reverse | TCTGACCAGGAGCTCCATTTTC | | |
| COL1A2 | 37 | Forward | CACTGGTGATCCTGGCAAAAAC | 134 | 60 |
| | 38 | Reverse | CACCTTTTCCACCTTGAACACC | | |
| COL3A1 | 39 | Forward | GTCAAGGGTGAAAGTGGGAAAC | 191 | 60 |
| | 40 | Reverse | GAGCCATTTTCACCACGATCAC | | |
| COL5A1 | 41 | Forward | CAAGTGGCACAGAATTGCTCTC | 130 | 60 |
| | 42 | Reverse | CACGATGATGCCATTGATGTCG | | |
| GJA1 | 43 | Forward | TCCCCTCTCGCCTATGTCTC | 100 | 60 |
| | 44 | Reverse | GTTTTGCTCACTTGCTTGCTTG | | |

### Immunocytochemistry

Cells were plated on iMatrix-511-coated plates and fixed in 4% paraformaldehyde (Nacalai, Kyoto, Japan) for 15 minutes. Cells were permeabilized with 0.1% Triton X-100 (Sigma-Aldrich, St. Louis, MO, United States)/PBS and blocked with 10% goat serum (Sigma-Aldrich). Primary and secondary antibodies were diluted in 0.1% Triton X-100/PBS with 5% goat serum and stained with Hoechst 33342 (Thermo Fisher Scientific) at 1 μg/mL, mouse anti-cardiac troponin T monoclonal IgG1 (GTX28295, GeneTex, Irvine, CA, United States) diluted at 1:16000, mouse anti-α-actinin monoclonal IgG1 (A7811, Sigma-Aldrich) diluted at 1:1000, rabbit anti-MLC2v monoclonal IgG (ab92721, Abcam, Cambridge, United Kingdom) diluted at 1:500, rabbit anti-CXCR4 monoclonal IgG (ab181020, Abcam) diluted at 1:2000, rabbit anti-NKX2-5 monoclonal IgG (Cat #. 8972S, Cell Signaling Technology, Danvers, MA, United States) diluted at 1:2500, mouse anti-Nav1.5 (Car #. ASC-013, Alomone Labs) diluted at 1:1000, rabbit anti-connexin 43 (Cat #. 83649, Cell Signaling Technology) diluted at 1:1000, and mouse anti-TBX5 monoclonal IgG2a (Cat #. sc-515536, Santa Cruz Biotechnology, Dallas, TX, United States) diluted at 1:500. These cells were then incubated with a secondary antibody, goat anti-mouse or rabbit polyclonal IgG conjugated with iFluor 488 or 555 (AAT Bioquest, Sunnyvale, CA, United States) diluted at 1:1000, for 1 hour at room temperature. The cells were then observed with IX71 (Olympus, Tokyo, Japan) or EVOS FL Auto (Thermo Fisher Scientific) and images processed with ImageJ (Scheneider et al., 2012, Nat Methods, 9:671-5).

### Flow Cytometry

Cells were dissociated with StemPro Accutase and stained with a LIVE/DEAD Fixable Green Dead Cell Stain Kit for 488 nm excitation (Thermo Fisher Scientific), and thereafter fixed with 1% paraformaldehyde for 15 minutes at room temperature. The cells were then permeabilized with 90% cold methanol for 30 minutes on ice and stained with mouse anti-cardiac troponin T monoclonal IgG1 (GTX28295, GeneTex) at 0.05 μg/100 μL overnight at 4° C. Goat anti-mouse or rabbit polyclonal IgG conjugated with Alexa Fluor 647 diluted at 1:2000 was used as a secondary antibody. The cells were resuspended in 0.5% bovine serum albumin (Merck)/PBS and analyzed with an Attune NxT Flow Cytometer (Thermo Fisher Scientific).

### Western Blot Analysis

Using the cell lysis solution contained in the Minute Total Protein Extraction Kit for Animal Cultured Cells/Tissues (Invent Biotechnologies, Plymouth, MN, United States) with Protease Inhibitor Cocktail Set III (×100) (Fujifilm Wako Pure Chemical Corporation, Osaka, Japan) and Phosphatase Inhibitor Cocktail Solution II (×100) (Fujifilm Wako Pure Chemical Corporation), cells at day 3 of differentiation were lysed and proteins were extracted according to the manufacturer's instructions. Protein concentration was measured with a Pierce BCA Protein Assay Kit (Thermo Fisher Scientific), iMark microplate reader and Microplate Manager 6 software (Bio-Rad, Hercules, CA, USA). Fifty micrograms of protein combined with Laemmli sample buffer (Bio-Rad) were boiled at 95° C. for 5 minutes and then loaded onto 10% Mini-PROTEAN TGX Precast Gels (Bio-Rad). Proteins were transferred to nitrocellulose membranes and blocking was performed using Tris buffered saline with Tween20 with 5% nonfat dry milk for 1 hour at room temperature. The membranes were probed with the following antibodies overnight at 4° C.: anti-SMAD1 (Cat. #. 6944S, Cell Signaling Technology) diluted at 1:1000, anti-phosphor-SMAD1/5 (Cat. #. 9516S, Cell Signaling Technology) diluted at 1:1000, anti-SMAD2/3 (Cat. #. 8685S, Cell Signaling Technology) diluted at 1:1000 and anti-phospho-SMAD2/3 (Cat. #. 8828S, Cell Signaling Technology) diluted at 1:1000. A horseradish peroxidase-conjugated secondary antibody was used to detect antibody binding. To enhance the signal, Easy-Western II super (Beacle, Kyoto, Japan) was used according to the manufacturer's instructions. Immunoblotted proteins were detected by ECL Prime Western blotting detection reagents and ImageQuant LAS4000 mini (GE Healthcare, Chicago, IL, USA).

### Measurement of Intracellular Ca²⁺-Transients

Cardiomyocytes were dissociated with TrypLE Select Enzyme (10×) (Thermo Fisher Scientific) and replated onto iMatrix-511-coated glass-based dishes (AGC Techno Glass, Shizuoka, Japan) at 1×10⁵/cm² on day 20. On day 34, the cells were incubated with 20 μM Fura 2-AM (Dojindo Laboratories, Kumamoto, Japan) for 30 minutes in a CO₂ incubator to load the indicator in the cytosol and then washed twice with Tyrode's solution. Fura 2-AM-loaded cells were alternately excited at 340 and 380 nm using a Lambda DG-4 Ultra High-Speed Wavelength Switcher (Sutter Instruments) coupled to an inverted IX71 microscope with a UApo 20×/0.75 objective lens (Olympus). Fura 2 fluorescent signals were recorded (ORCA-Flash 2.8; Hamamatsu Photonics) during 0.2 Hz field stimulation and analyzed by a ratiometric fluorescence method using MetaFluor software (version 7.7.5.0; Molecular Devices).

### Screening Compounds for Treatment of RV-Related Disease

hPSCs from patients with RV dysfunction related diseases such as Arrhythmogenic cardiomyopathy, Brugada syndrome, or congenital heart disease (Tetralogy of Fallot, Ebstrein Anomoly, Single left ventricle, pulmonary hypertension) are isolated and used to differentiate into RV cardiomyocytes as described above. Candidate compounds are cultured with these RV cardiomyocytes. The functionality of RV cardiomyocytes in the presence and absence of each compound are measured by contractility, calcium transient, action potential, and field potential.

Transplantation of iPSC-RV Cells

Immunodeficient rats are open chested and an aluminum rod chilled with liquid nitrogen is pressed against the right ventricle to injure the heart (cryoinjury model). 5×10⁶ of iPSC-RV cells or 5×10⁶ of SHF cardiac progenitor cells are delivered via intramyocardial injection to the site of RV injury. The RV and SHF cells are derived from PSC using the described methods with insulin or DM addition. Control animals are treated with cardiomyocytes and cardiac progenitor cells that are generated from PSCs without insulin or DM addition; or treated without any cells (RPMI/B27 minus insulin). Several parameters are measured and compared between groups: the size and percentage of fractional shortening of the RV (as measured by electrocardiography), RV pressure (measured by right hear catheterization), fibrosis of the RV and retention of injected RV cardiomyocytes/SHF cardiac progenitor cells in the RV (as measured by immunohistochemistry), and arrhythmia (as monitored by implantable telemetry device).

### Statistical Analysis

All data are expressed as means±standard deviation (SD). Statistical analysis was performed by Student's t-test using SPSS statistics 24 (IBM, Armonk, NY, United States). Values of P<0.05 were considered to be significant.

### Example 1

### Insulin Promotes Differentiation of SHF Cardiac Progenitors in the Presence of Wnt Signaling

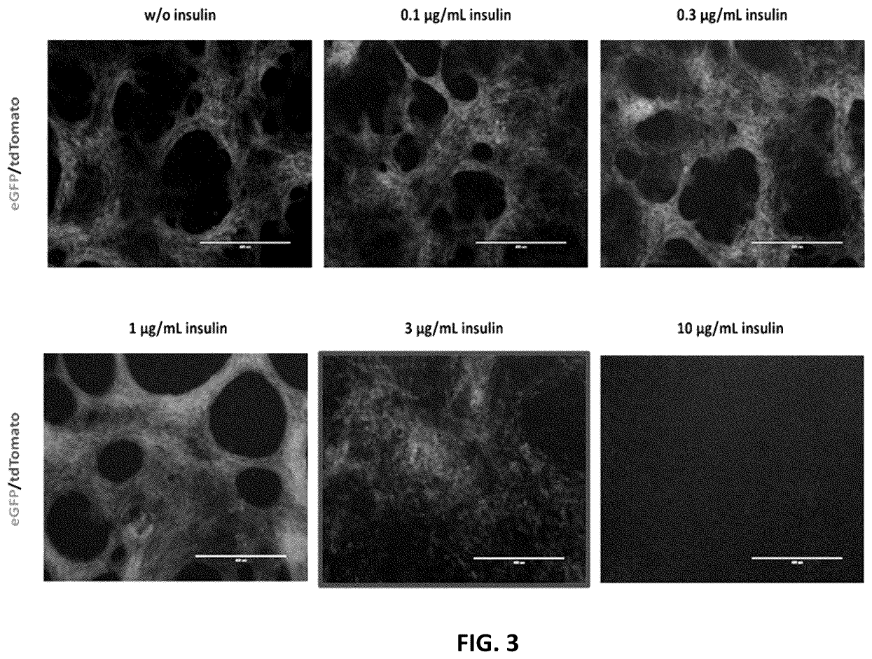

Prior studies have demonstrated that insulin signaling inhibits formation of cardiac mesoderm in some protocols using embryoid bodies, conditioned medium, and Activin A/BMP-4 mediated differentiation (Lian et al., 2013, Stem Cells 31:447-57); nevertheless, insulin did not inhibit cardiac differentiation in a small molecule protocol using biphasic modulation of Wnt signaling (GiWi protocol). However, prior studies did not examine the impact of insulin on the type of cardiac progenitors that form in the small molecule Wnt modulation protocol. Using a HES3-TBX5-TdTomato^{+/W}/NKX2-5^{eGFP/W} reporter hESC line that can identify FHF (TBX5-TdTomato⁺/NKX2-5-eGFP⁺) and SHF (TBX5-TdTomato⁻/NKX2-5-eGFP⁺) cardiac progenitors, B-27 supplement with and without insulin was tested on differentiation day 0 (FIG. 2A). Strong TBX5-tdTomato expression was detected at the cardiac progenitor stage on day 7 by fluorescence microscopy in the absence of insulin but not in its presence (FIG. 2B). Subsequently, both control and insulin-treated cells expressed NKX2-5-eGFP, but TBX5-tdTomato expression level in insulin-treated cells was lower than that in control cells on day 14, corresponding to the cardiomyocyte stage (FIG. 2C). Because the insulin concentration contained in the B-27 supplement is not disclosed, several concentrations of insulin were tested and it was found that 3 μg/mL insulin resulted in strong Nkx2.5-eGFP expression without TBX5-tdTomato expression on day 13 consistent with the genesis of SHF-derived hPSC-CMs (FIG. 3A).

Insulin treatment (with B-27-containing insulin) also was tested from day 0 to day 1 in a reporterless hiPSC line to confirm the impact of insulin in an independent line (FIG. 4A). Insulin-treated cells showed lower expression levels of FHF markers (TBX5 and HCN4) and higher mRNA expression levels of SHF markers (ISL1 and CXCR4) than in control cells on day 7 (FIG. 4B). Immunostaining for CXCR4 confirmed its upregulation (FIG. 4C). Both control and insulin-treated cells expressed pan-cardiomyocyte markers, NKX2-5 and cardiac Troponin T (cTnT) on day 10 (FIG. 4D). Most of the control cells expressed TBX5, while TBX5 expression was greatly reduced in insulin-treated cells (FIG. 4E). Together, these findings indicated that adding insulin at the initiation of hPSC differentiation (day 0) in the GiWi protocol unexpectedly led to formation of SHF cardiac progenitors in contrast to predominantly FHF cardiac progenitors in the absence of insulin.

### Example 2

### Insulin Suppresses Activation of BMP Signaling

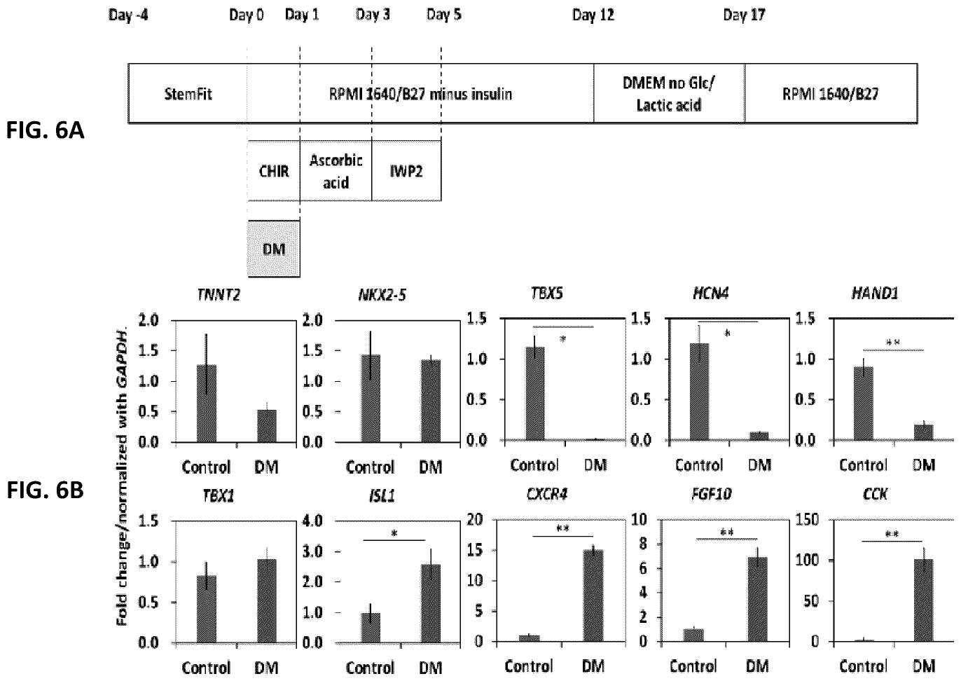

Previous studies demonstrated that insulin inhibited cardiac differentiation of hPSCs in Activin A/BMP4-based protocols but not in the Gsk3 inhibitor/Wnt inhibitor (GiWi) protocol (Lian et al., 2013, Stem Cells 31(3):447-57; Xu et al., 2008, Differentiation 76(9):958-70). Consequently, phosphorylation of SMADs in GiWi protocol was investigated which can be used to indicate Activin A (SMAD2/3 phosphorylation) and BMP4 activation (SMAD 1/5 phosphorylation). Insulin added on differentiation day 0 suppressed phosphorylation of SAMD1/5 but not phosphorylation of SAMD2/3 measured on day 3 (FIGS. 5A and 5B). These results indicated that insulin inhibited activation of intrinsic BMP signaling during mesoderm induction.

### Example 3

Inhibition of BMP Signaling Induced SHF Cells from Reporterless hiPSCs

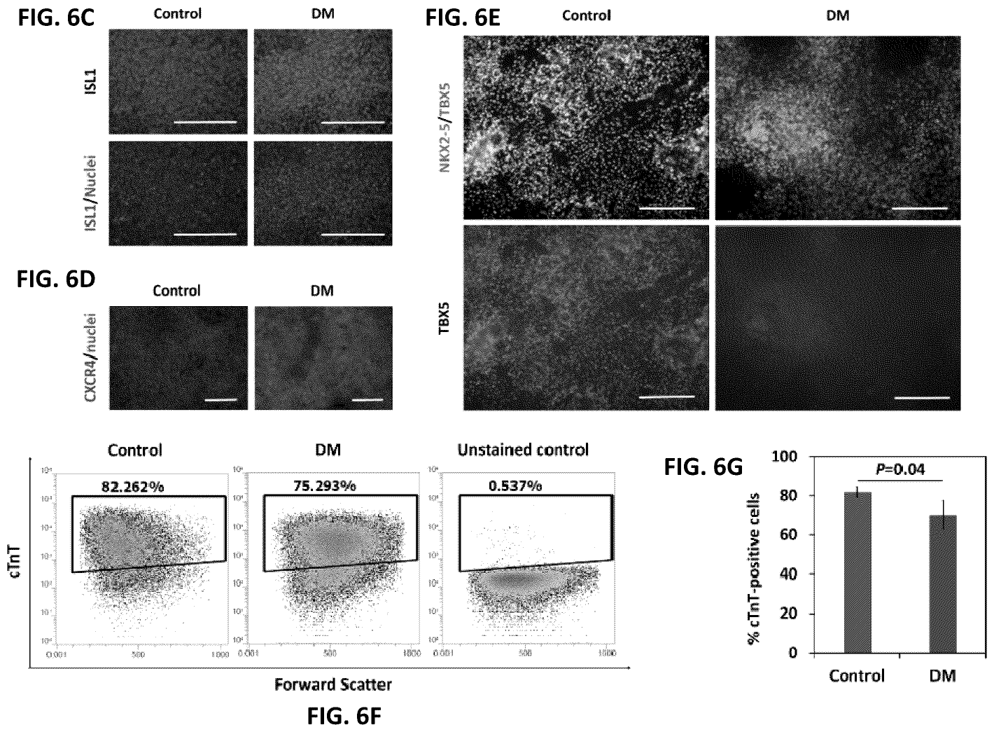

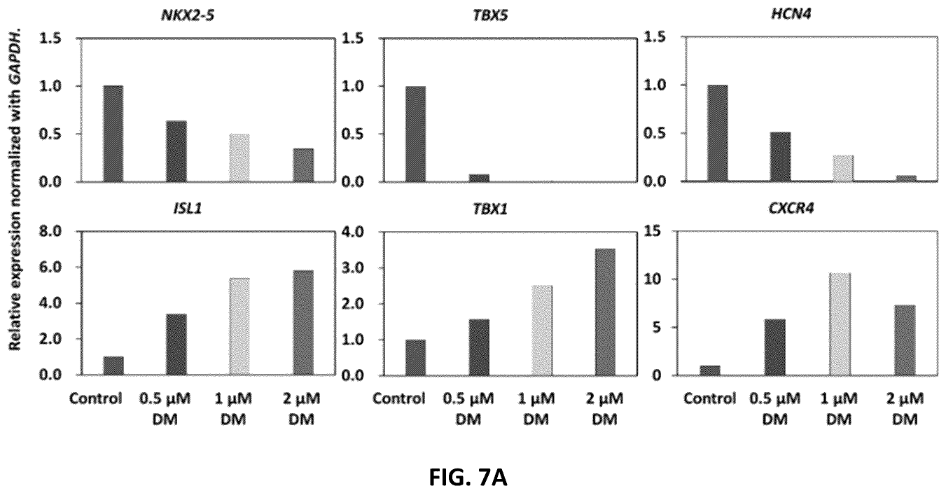

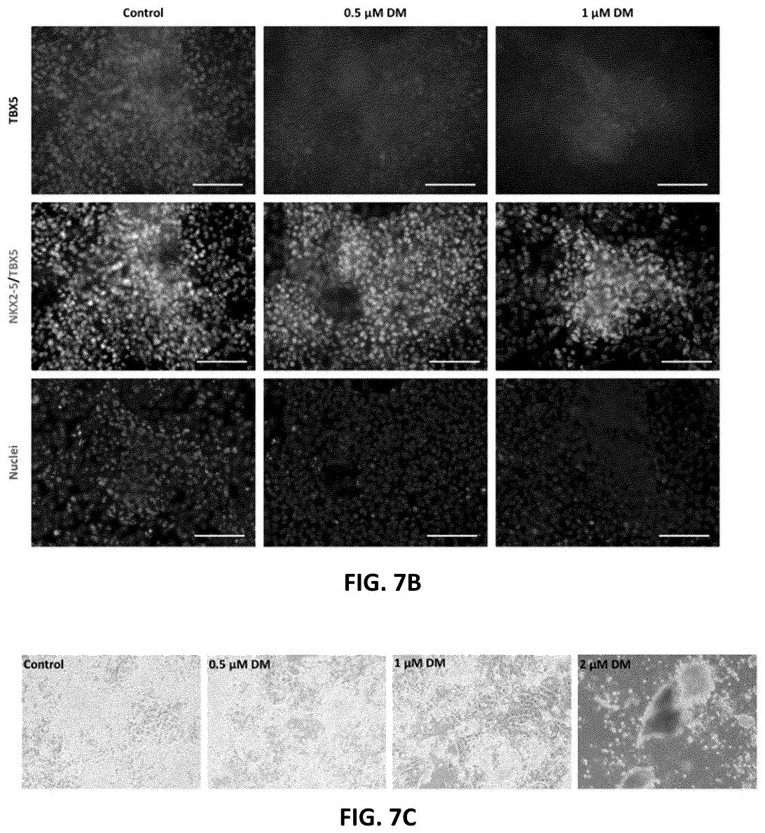

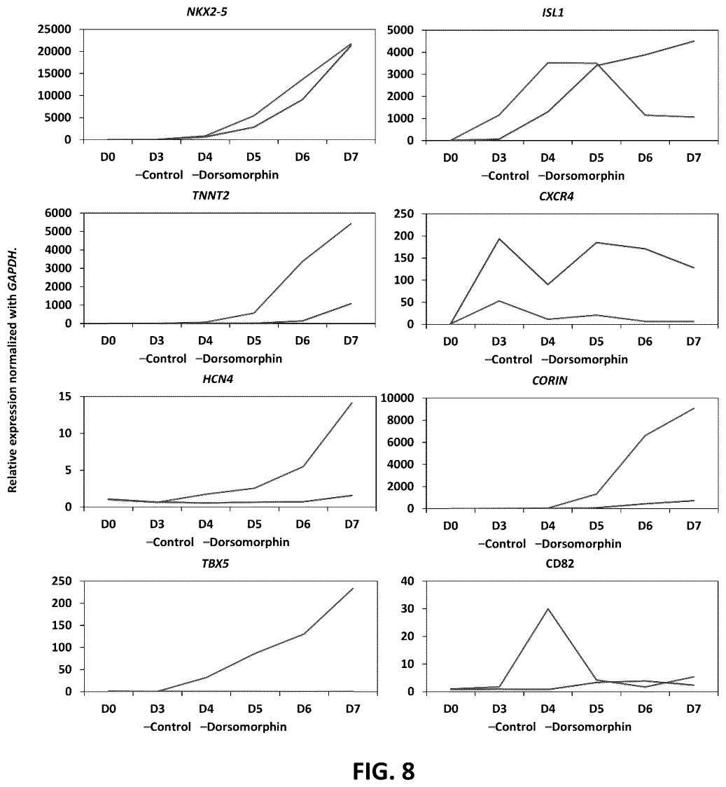

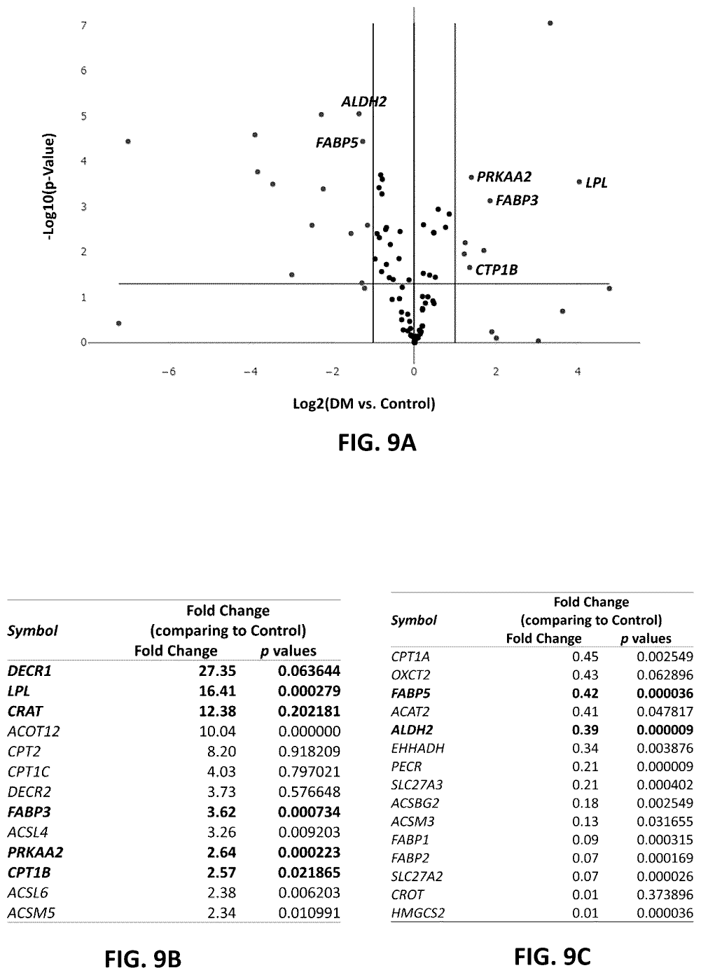

To determine if inhibiting BMP signaling directly could mimic the insulin effect on cardiac progenitor formation, the effect of different concentrations of the BMP antagonist, dorsomorphin, added on differentiation day 0 (FIG. 6A) was tested. In these experiments, dorsomorphin reduced expression of FHF markers (TBX5 and HCN4) and enhanced expression of SHF markers (TBX1, ISL1 and CXCR4) measured on day 7 (FIG. 6B) in a dose-dependent manner (FIGS. 7A and 7B). However, 2 μmol/L dorsomorphin-treated cells did not survive metabolic selection using no glucose medium supplemented with lactic acid (FIG. 7C). This suggested that cardiomyocytes were not induced in 2 μmol/L dorsomorphin-treated cells. Therefore, 1 μmol/L dorsomorphin was used for subsequent experiments. To evaluate the temporal pattern of changes in gene expression in response to dosomorphin added on day 0, quantitative polymerase chain reaction (q-PCR) analysis was performed from day 0 to day 7 for a series of cardiomyocyte and cardiac progenitor genes (FIG. 8). Dorsomorphin completely suppressed upregulation of TBX5 until day 7. ISL1 was highly expressed in control cells until day 5 then its expression level declined. On the other hand, gradual upregulation of ISL1 was seen until day 7 in dorsomorphin-treated cells. Expression of CORIN, a FHF marker (Zhang et al., 2019, Cell Stem Cell 24:802-811.e5.; Pezhouman et al., 2022 Cardiovasc Res. 118:828-43), and expression of CD82, a marker of cardiomyocyte-fated progenitors (Takeda et al., 2018, Cell Rep. 22:546-56), were also suppressed in dorsomorphin-treated cells. Expression of NKX2-5 and TNNT2, pan-cardiac markers, was delayed in dorsomorphin-treated cells.

Immunolabeling showed that both control and dorsomorphin-treated cells expressed ISL1 protein on day 6 and that CXCR4 protein was highly expressed in dorsomorphin-treated cells on day 7 (FIGS. 6C and 6D). Both control and dorsomorphin-treated cells expressed NKX2-5 protein, while TBX5 protein expression was reduced in dorsomorphin-treated cells on day 10 (FIG. 6E). On day 12, the percentages of cTnT-positive cells were 82.1±2.6% in control cells and 70.4±7.3% in dorsomorphin-treated cells (p=0.04) (FIGS. 6F and 6G). Interestingly, cTnT-positive cells in dorsomorphin-treated cells showed larger forward scatter than control cells did, suggesting that the size of cardiomyocytes in dorsomorphin-treated cells was larger than that in control cells (FIG. 6F).

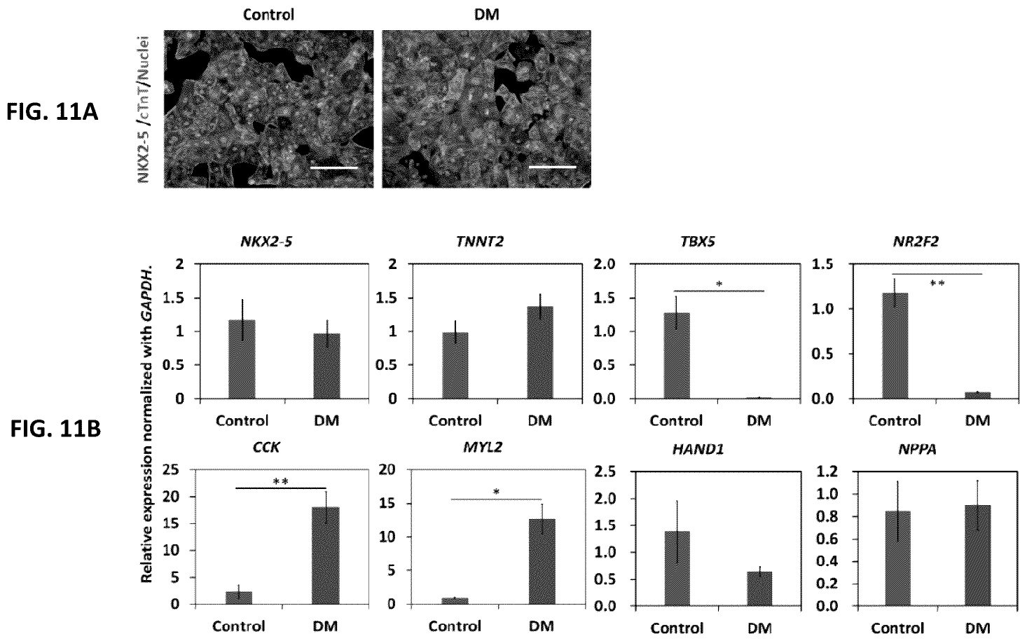

Dorsomorphin was known in the art to inhibit not only BMP signaling but also AMPK signaling. Therefore, DMH1, a selective inhibitor of the BMP receptor, was also tested. Treatment with DMH1 on differentiation day 0 also reduced TBX5 expression in a dose-dependent manner and induced NKX2-5-positive/cTnT-positive cardiomyocytes (FIGS. 10A and 10B). Treatment with 0.5 μmol/L DMH1 upregulated CXCR4 expression on day 7 (FIG. 10C).

### Example 4

RV Myocytes Arose from SHF Cells

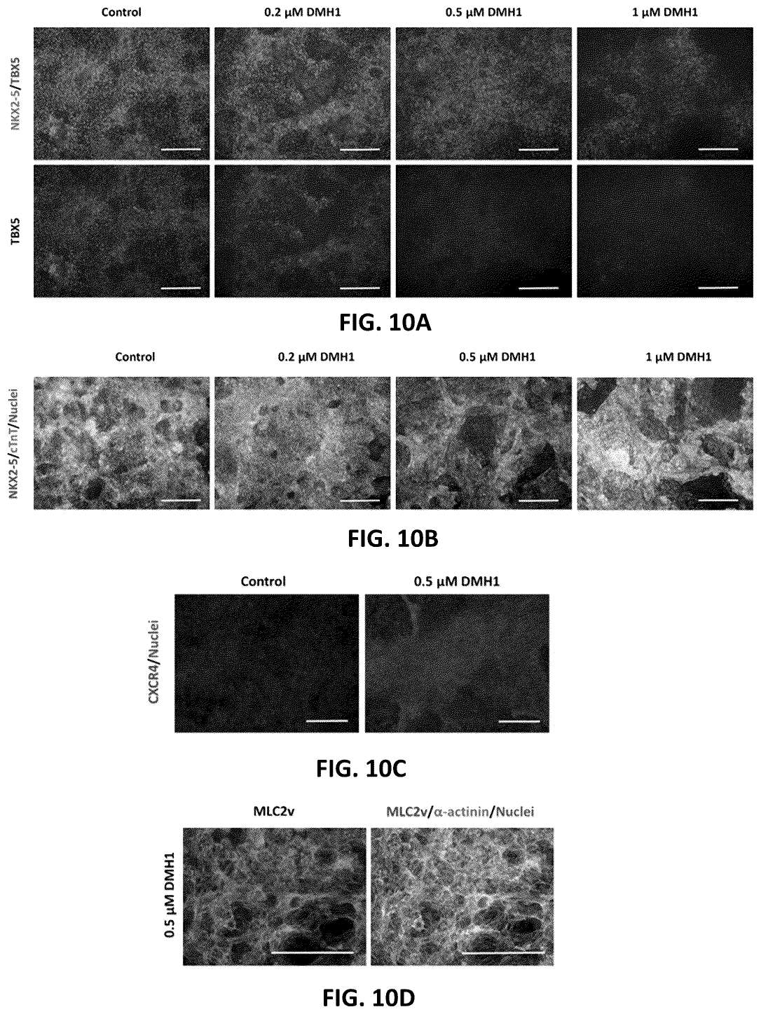

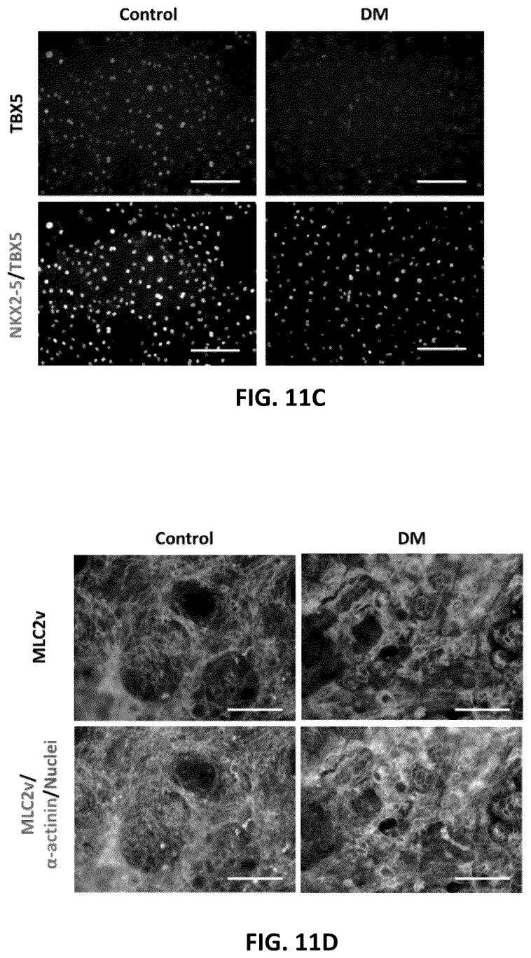

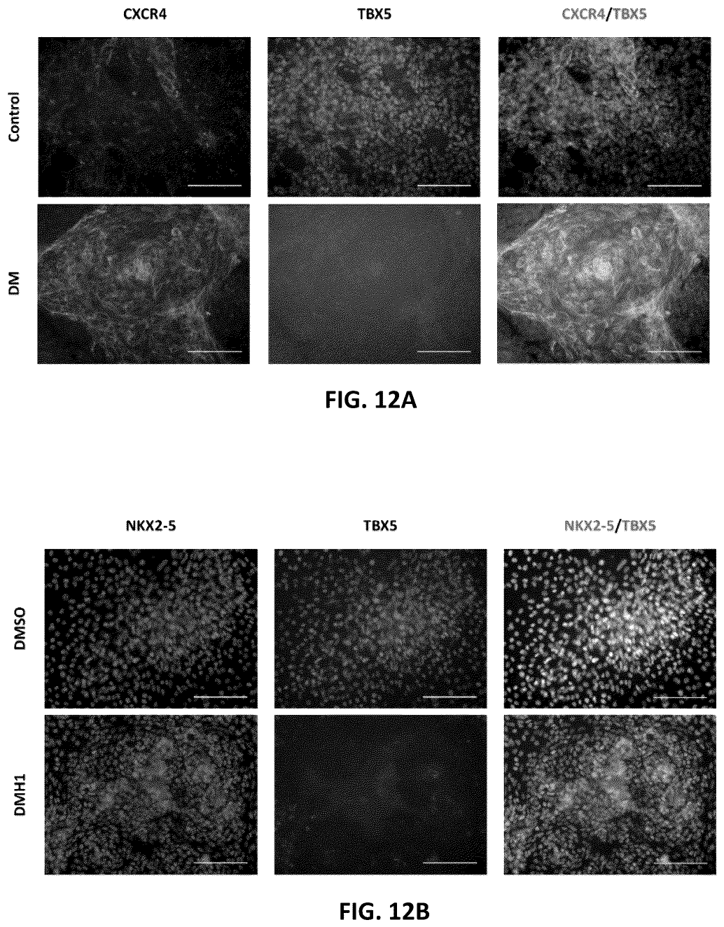

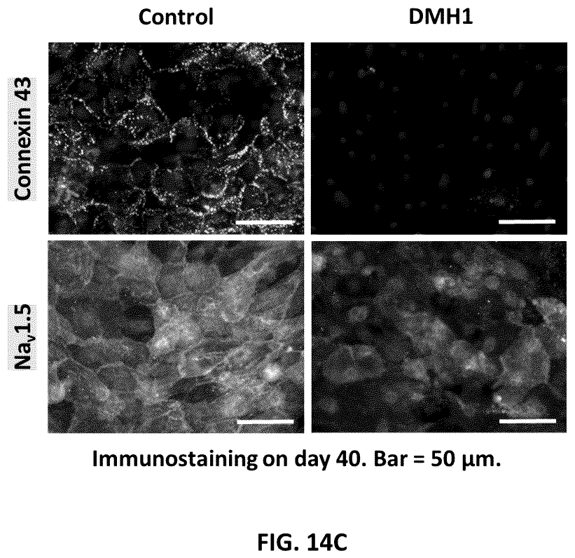

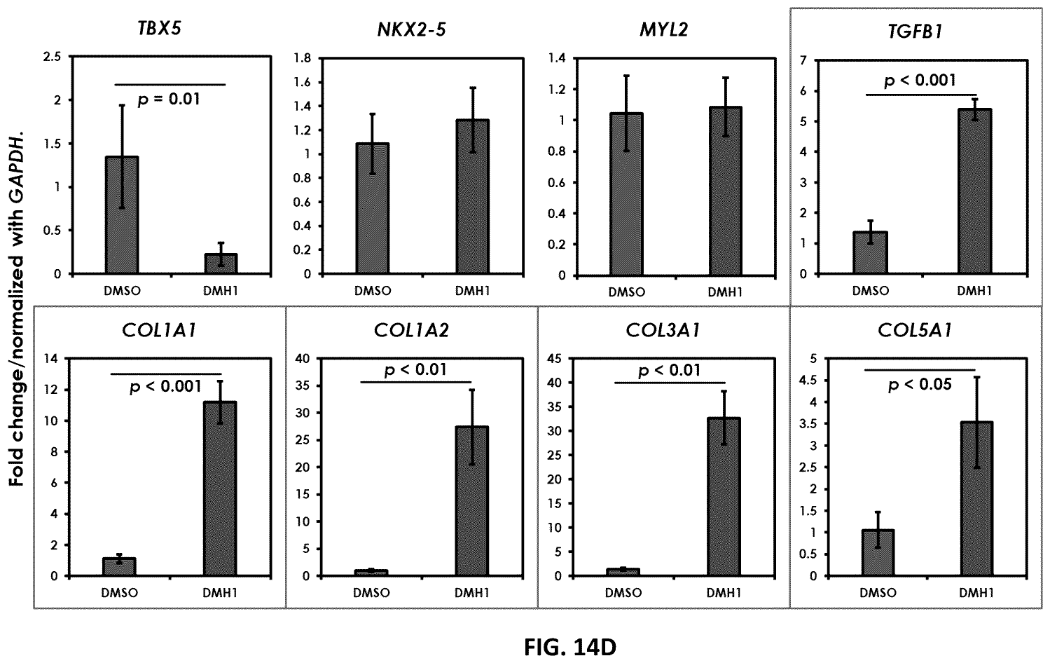

Cardiomyocytes were purified by culture in glucose-free/lactic acid supplemented medium (see Tohyama et al., 2013 Cell Stem Cell 12:127-137). Most of the cells in both the control and dorsomorphin-treated groups expressed NXK2-5 and cTnT as shown in FIG. 11A. Quantitative PCR data also showed that there was no significant difference between the two groups in expression of cardiomyocyte markers, NKX2-5 and TNNT2 and a working cardiomyocyte marker, NPPA (Horsthuis et al., 2008, Circ Res. 102:849-59). However, expression of CCK (RV marker) (De Soysa et al., 2019, Nature 572:120-24) and MYL2 (ventricular marker) was significantly upregulated and expression of TBX5 (LV and atrial marker) and NR2F2 (atrial and nodal marker) were significantly downregulated in hPSC-CMs derived from dorsomorphin-treated cells. Expression levels of HAND1 (LV marker) trended to be lower with dorsomorphin treatment (FIG. 11B). Immunolabeling also showed less TBX5 expression in cardiomyocytes from dorsomorphin-treated cells (FIG. 11C). At a later differentiation stage (day 35), both groups of cardiomyocytes expressed MLC2v protein, a ventricular-type myosin light chain isoform (FIG. 11D). Comparison of expression of 84 genes involved in fatty acid metabolism showed that the expression levels of LPL, FABP3, PRKAA2 and CPT1B genes involved in cardiac fatty acid metabolism were higher in dorsomorphin-treated cell-derived cardiomyocytes than those in control cardiomyocytes (FIG. 9A to FIG. 9C). It has been shown that GJA1 expression encoding Connexin 43 is lower in the right ventricle of the human embryonic heart than in the left ventricle (Cui et al., 2019, Cell Rep. 26(7): 1934-1950). It has also been reported that GJA1 and SCN5A expression (SCN5A encoding Naᵥ1.5) is lower in the right ventricular outflow tract of rabbits and mice (Ou et al., 2005, Life Sci. 77(1): 52-9; Boukens et al., 2013, Circ Res. 113(2): 137-141). The experimental data showed that DM-treated cell-derived cardiomyocytes showed less gene expression of SCN5A and GJA1 (FIG. 14A) which were further supported by reduction in immunostaining signal of Naᵥ1.5 and Connexin 43 in both DM-treated and DMH1-treated cell-derived cardiomyocytes (FIG. 14B and FIG. 14C). Naᵥ1.5 and Connexin 43 are encoded by SCN5A and GJA1 respectively. It is reported that collagen deposition is more common in the right ventricle of adult humans than in the left ventricle (Miles et al., 2021, J Am Coll Cardiol. 78(15): 1511-1521); thus, a panel of extracellular matrix and collagen-related gene expression of TGFB1, COL1A1, COL1A2, COL3A1, and COL5A1 was tested and the results showed that expression of all of these genes was reduced in DMH1-treated cell-derived cardiomyocytes at day 34.

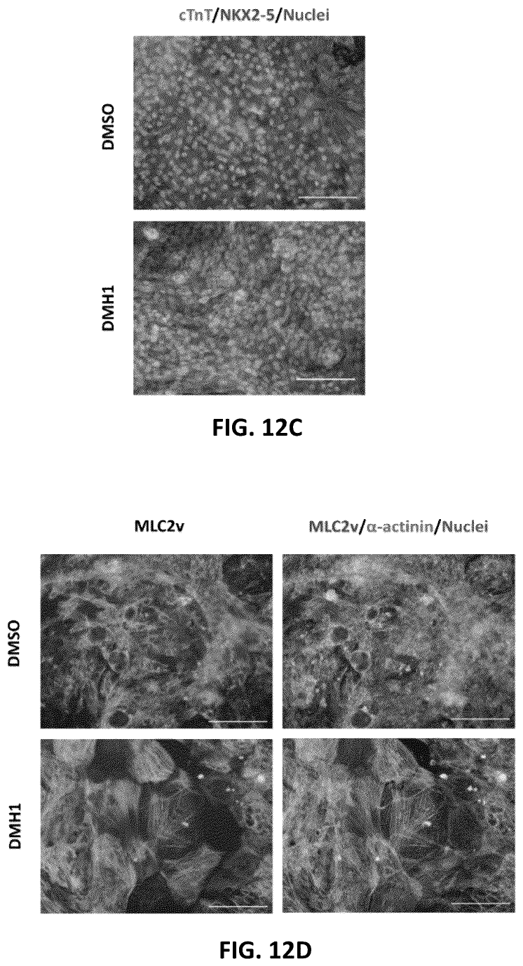

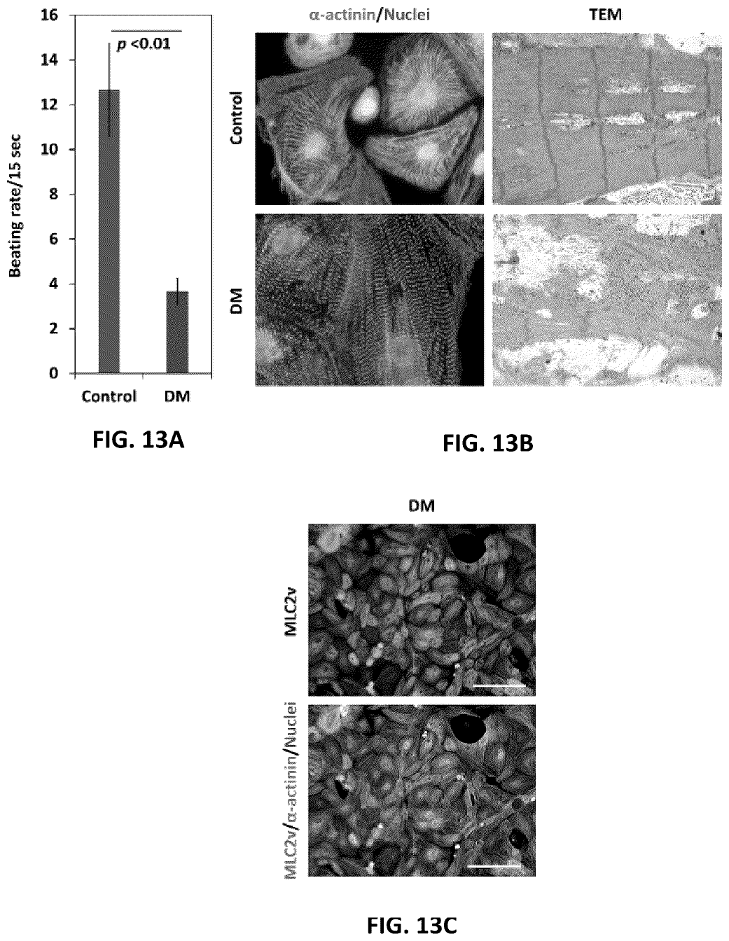

Additionally, cardiomyocytes derived from 0.5 μmol/L DMH1-treated cells also expressed MLC2v, a ventricular marker protein (FIG. 10D). As shown in FIG. 12, similar results were obtained using commercially available hiPSC line, 201B7.

### Example 5

Time restraint for addition of insulin and DM/DMH1 to induce RV cardiomyocyte differentiation

To determine the optimal time window for addition of insulin or DM/DMH1 to induce differentiation of RV cardiomyocytes, DM or insulin was added on day 0, day 1, day 3, or day 5 of PSC differentiation. FIG. 15A showed that beyond day 5, DM failed to induce RV cardiomyocytes. FIG. 15B showed that insulin induced optimal RV cardiomyocytes differentiation if it was added on day 1 for 48 hours. Note that the same result could be obtained when insulin was added on day 0 for 24 hours (FIG. 4A through FIG. 4E)

### Example 6

Phenotypic Characteristics of RV Myocytes Derived from hiPSCs

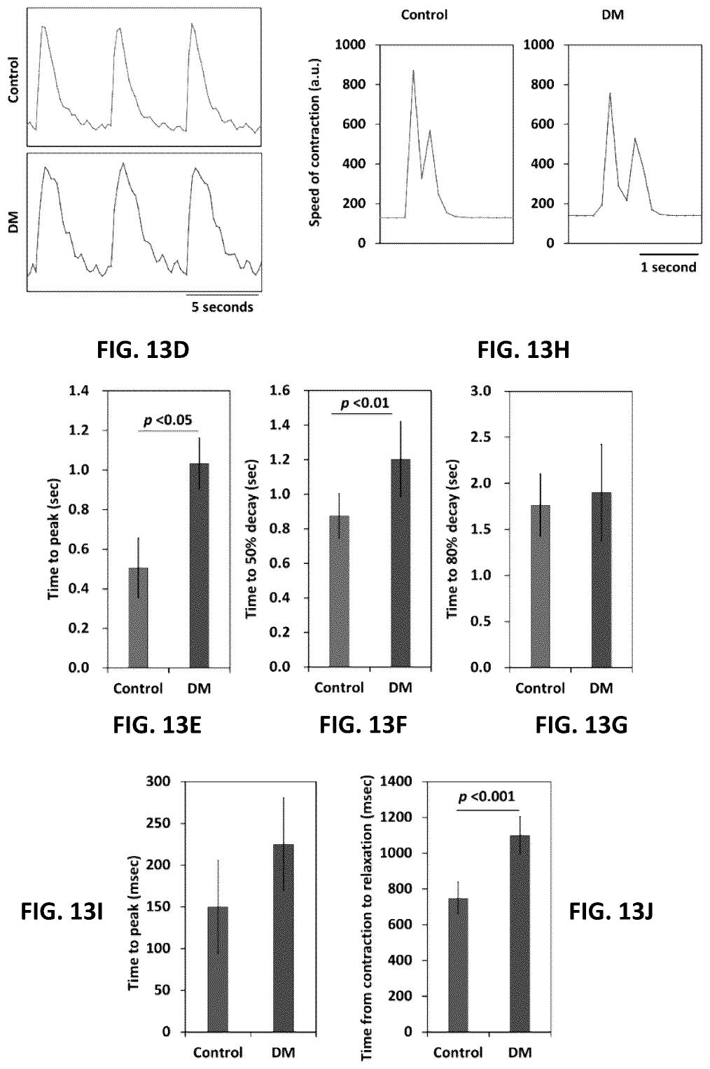

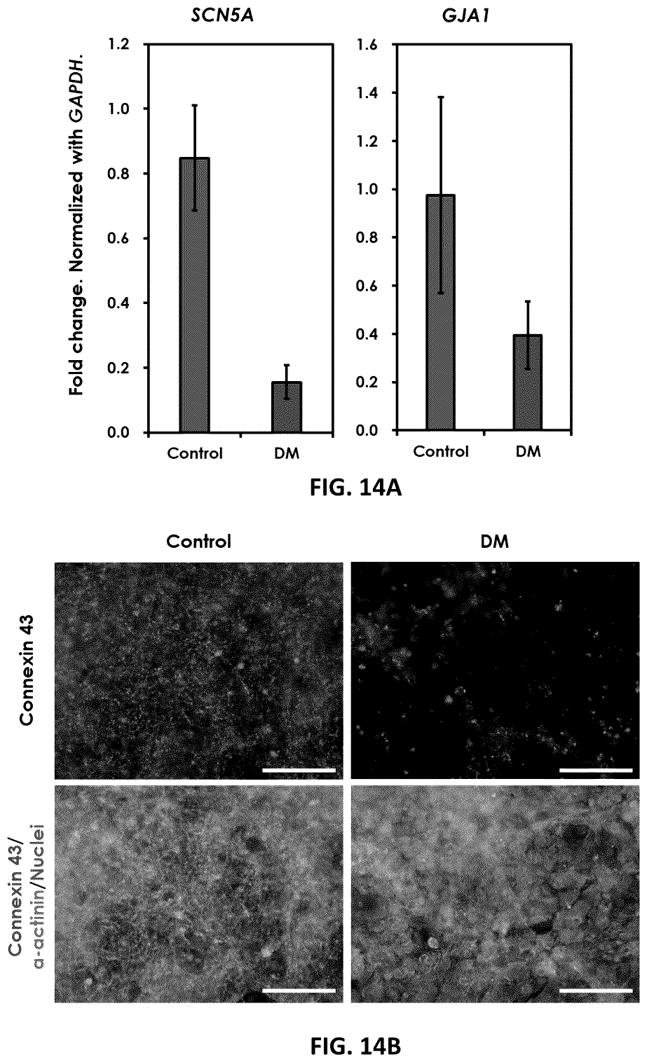

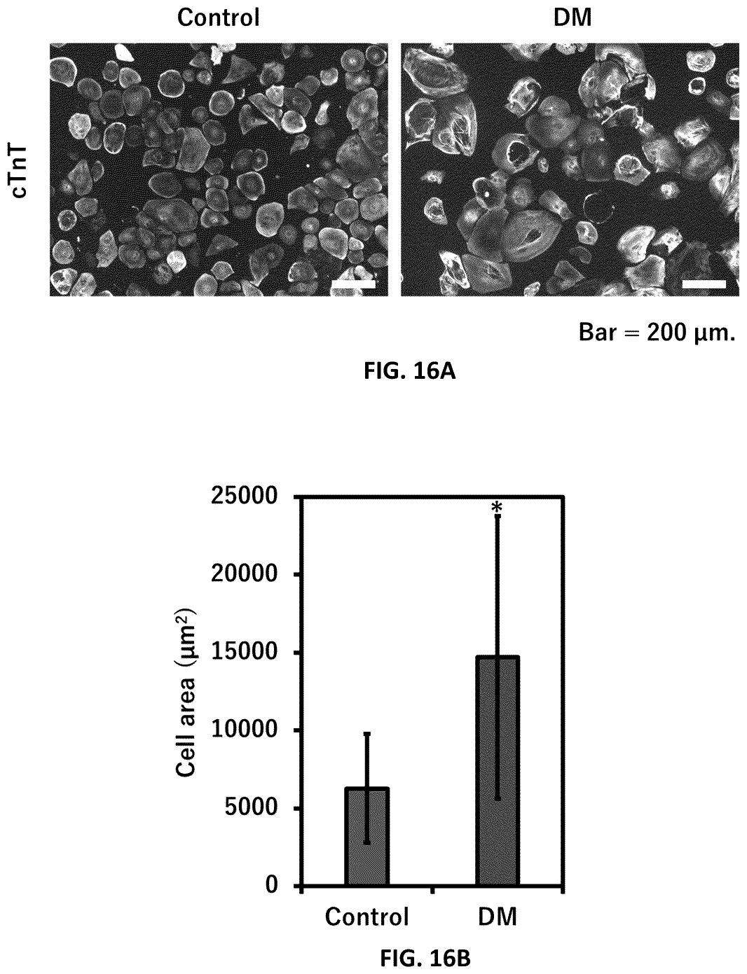

Spontaneous contracting rates of cardiomyocytes from dorsomorphin-treated cells were lower than those of control cardiomyocytes (FIG. 13A). The morphology of dorsomorphin-treated cell-derived cardiomyocytes was flat and spread out compared to control cardiomyocytes, and immunostaining and electron microscopic findings showed that dorsomorphin-treated cell-derive cardiomyocytes had poor sarcomere formation, even though the mature ventricular marker MLC2v was robustly expressed (FIGS. 13B and 13C). Calcium transients in both groups of cardiomyocytes are shown in FIG. 13D, wherein time to peak and time to 50% decay of the calcium transient were longer in cardiomyocytes from dorsomorphin-treated cells than in control cardiomyocytes (FIG. 13E to FIG. 13G). The contraction speeds of cardiomyocytes are shown in FIG. 13H, wherein time to peak contraction tended to be longer in cardiomyocytes from dorsomorphin-treated cells than in control cardiomyocytes (FIG. 13I). The time from the start of contraction to relaxation was significantly longer in cardiomyocytes from dorsomorphin-treated cells than in control cardiomyocytes (FIG. 13J). Additionally, cardiomyocytes derived PSCs cells treated with DM were significantly larger in size compared to control cells (FIG. 16A and FIG. 16B) which is consistent with RV cardiomyocytes' characteristics in comparison to LV cardiomyocytes.

### Example 7

Compound Screening with RV Cardiomyocytes Differentiated from hPSCs Derived from Patients with RV Dysfunction Related Disease

To test whether the RV cardiomyocytes can be used as model RV disease in vitro, RV cardiomyocytes differentiated from hPSCs derived from patients with RV dysfunction related disease are generated and incubated with candidate compounds. The results show that RV cardiomyocytes incubated with candidate compounds have superior conduction velocity, lower frequency of arrhythmia and lower frequency of abnormal calcium transient compared to RV cardiomyocytes incubated with DMSO.

### Example 8

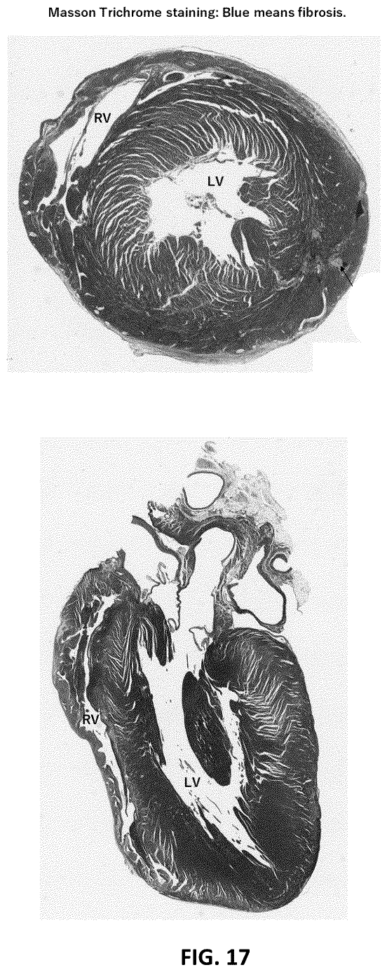

Transplantation of iPSC-RV Cells into Immunodeficient Rats
RV and LV cardiomyocytes have different electrical properties (Bernal-Ramirez et al., 2021, Oxid Med Cell Longev. 2021(9993060): 21 pages). This raises concerns about proarrhythmic effects due to cardiomyocyte heterogeneity after cell transplantation. Therefore, transplantation of separately produced iPSC-derived or ESC-derived LV and RV cells into the respective ventricles may be useful to overcome this concern. RV cardiomyocytes or SHF cardiac progenitor cells are injected intramyocardially into the site of injury in the heart of immunodeficient rats. The RV of the rat is injured specifically with an aluminum rod cooled with liquid nitrogen (cryoinjury model). The injury is shown in blue in FIG. 17. The results show that treated mice have reduced injury size (as shown by reduced fibrosis sites), enhanced contractility (as shown by higher fractional shortening and RV pressure), less frequent ventricular tachycardia compared to animals treated with cardiac progenitor cells or cardiomyocytes derived from PSC using methods without insulin or DMH addition, or animals treated without any cells (RPMI/B27 minus insulin). Furthermore, the immunostaining of heart tissues shows high retention of the injected RV cardiomyocytes/SHF cardiac progenitor cells.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

We claim:

1. A method for producing a population of right ventricular (RV)-like cardiomyocytes from human pluripotent stem cells (hPSCs) comprising:
a) culturing hPSCs with a glycogen synthase kinase 3 (Gsk3) inhibitor to obtain a first population of cells;
b) thereafter optionally culturing the first population of cells without the Gsk-3 inhibitor for a period of time;
c) culturing the first population of cells with a Wnt/β-catenin signaling pathway inhibitor to obtain a second population of cells comprising second heart field (SHF) cardiac progenitor cells; and
d) culturing the second population of cells comprising SHF cardiac progenitor cells without Wnt/β-catenin signaling inhibitor to obtain a third population of cells comprising RV cardiomyocytes,
wherein an insulin signaling activator is added anytime between step (a) and step (b).

2. The method of claim 1, wherein the insulin signaling activator is present in culture media for 24 to 48 hours.

3. The method of claim 1, wherein the insulin signaling activator is insulin, IGF-1, S597 or a combination thereof.

4. The method of claim 1, wherein the Gsk-3 inhibitor is CHIR 99021, CHIR 98014, BIO-acetoxime, BIO, LiCl, SB 216763, SB 415286, AR A014418, 1-Azakenpaullone, and Bis-7-indolylmaleimide, or a combination thereof.

5. The method of claim 1, wherein the Wnt/β-catenin signaling pathway inhibitor is XAV939, IWR-1, IWR-2, IWR-3, IWR-4, IWR-5, IWP-1, IWP-2, IWP-3, or IWP-4.

6. The method of claim 1, wherein the SHF cardiac progenitor cells are between 70% and 99% of the second population of cells without cell separation or cell selection steps.

7. The method of claim 1, wherein the RV cardiomyocytes are between 60% and 80% of the third population of cells without cell separation or selection steps.

8. The method of claim 1, wherein the RV cardiomyocytes have distinct phenotypic properties compared to cardiomyocytes produced by the same method but without culturing the cells in the presence of the insulin signaling activator.

9. The method of claim 8, wherein the phenotypic properties are sarcomere formation, cell size, spontaneous contraction rate, Ca²¹ transient event duration, contraction speed, and relaxation speed.

10. A method for producing a population of right ventricular (RV)-like cardiomyocytes from human pluripotent stem cells (PSCs) comprising:
a) culturing hPSCs with a glycogen synthase kinase 3 (Gsk3) inhibitor to obtain a first population of cells;
b) thereafter optionally culturing the first population of cells without the Gsk-3 inhibitor;
c) culturing the first population of cells with a Wnt/β-catenin signaling pathway inhibitor to obtain a second population of cells comprising second heart field (SHF) cardiac progenitor cells; and
d) culturing the second population of cells comprising SHF cardiac progenitor cells without Wnt/β-catenin signaling inhibitor to obtain a third population of cells comprising RV cardiomyocytes
wherein a bone morphogenic protein (BMP) signaling inhibitor is added anytime between step (a) and step (c).

11. The method of claim 10, wherein the BMP signaling inhibitor is present in culture media for 24 to 48 hours.

12. The method of claim 10, wherein the BMP signaling pathway inhibitor is dorsomorphin, dorsomorphin homologue 1, LDN-193189, K02288, LDN-214117, ML347, LDN-212854 or a combination thereof.

13. The method of claim 10, wherein the Gsk-3 inhibitor is CHIR 99021, CHIR 98014, BIO-acetoxime, BIO, LiCl, SB 216763, SB 415286, AR A014418, 1-Azakenpaullone, and Bis-7-indolylmaleimide, or a combination thereof.

14. The method of claim 10, wherein the Wnt/β-catenin signaling pathway inhibitor is XAV939, IWR-1, IWR-2, IWR-3, IWR-4, IWR-5, IWP-1, IWP-2, IWP-3, or IWP-4.

15. The method of claim 10, wherein the SHF cardiac progenitor cells are between 70% and 99% of the second population of cells without cell separation or cell selection steps.

16. The method of claim 10, wherein the RV cardiomyocytes are between 60% and 80% of the third population of cells without cell separation or selection steps.

17. The method of claim 10, wherein the RV cardiomyocytes have distinct phenotypic properties compared to cardiomyocytes produced by the same method but without culturing the cells in the presence of the BMP signaling inhibitor.

18. The method of claim 17, wherein the phenotypic properties are sarcomere formation, cell size, spontaneous contraction rate, Ca²¹ transient event duration, contraction speed, and relaxation speed.

19. A method for screening a compound using right ventricular (RV) cardiomyocytes derived from hPSCs comprising:
a) culturing the RV cardiomyocytes with the compound;
b) culturing in parallel the RV cardiomyocytes without the compound;
c) measuring a functional parameter of the RV cardiomyocytes in (a) and in (b); and
d) comparing the measured functional parameter of the RV cardiomyocytes in (a) with the measured functional parameter of the RV cardiomyocytes in (b).

20. The method of claim 19, wherein the hPSCs are derived from patients that have RV dysfunction-related diseases.

21. The method of claim 20, wherein the RV dysfunction-related disease is Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, or Eisenmenger Syndrome.

22. A population comprising SHF cardiac progenitor cells produced by step a) to step c) of the method of claim 1.

23. A population comprising RV cardiomyocytes produced by the method of claim 1.

24. The population of claim 23, wherein the RV cardiomyocytes have distinct phenotypic properties compared to cardiomyocytes produced by the same method but without culturing the cells in the presence of the insulin signaling activator.

25. The population of claim 24, wherein the phenotypic properties are sarcomere formation, cell size, spontaneous contraction rate, Ca²¹ transient event duration, contraction speed, and relaxation speed.

26. A population comprising SHF cardiac progenitor cells produced by step a) to step c) of the method of claim 10.

27. A population comprising RV cardiomyocytes produced by the method of claim 10.

28. The population of claim 27, wherein the RV cardiomyocytes have distinct phenotypic properties compared to cardiomyocytes produced by the same method but without culturing the cells in the presence of BMP signaling inhibitor.

29. The population of claim 28, wherein the phenotypic properties are sarcomere formation, cell size, spontaneous contraction rate, Ca²¹ transient event duration, contraction speed, and relaxation speed.

30. A method of treating a subject with cardiac disease impacting the right ventricle comprising administering to the subject a pharmaceutical composition comprising the SHF cardiac progenitor cells produced by the method of claim 1.

31. The method of claim 30, wherein the cardiac disease impacting the right ventricle is Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, or Eisenmenger Syndrome.

32. A method of treating a subject with cardiac disease impacting the right ventricle comprising administering to the subject a pharmaceutical composition comprising the SHF cardiac progenitor cells produced by the method of claim 10.

33. The method of claim 32, wherein the cardiac disease impacting the right ventricle is Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, or Eisenmenger Syndrome.

34. A method of treating a subject with cardiac disease impacting the right ventricle comprising administering to the subject a pharmaceutical composition comprising the RV cardiomyocytes produced by the method of claim 1.

35. The method of claim 34, wherein the cardiac disease impacting the right ventricle is Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, or Eisenmenger Syndrome.

36. A method of treating a subject with cardiac disease impacting the right ventricle comprising administering to the subject a pharmaceutical composition comprising the RV cardiomyocytes produced by the method of claim 10.

37. The method of claim 36, wherein the cardiac disease impacting the right ventricle is Brugada syndrome, arrhythmogenic right ventricular cardiomyopathy, pulmonary artery hypertension, atrial septal defect, Ebstein's anomaly, Tetralogy of Fallot, tricuspid atresia, double outlet right ventricle, or Eisenmenger Syndrome.


SEQUENCE LISTING (44 sequences)

SEQ ID NO: 1 - RNA, 57 nt, synthetic construct
  source 1..57: mol_type = other RNA
ccggaggtgctatctgtctgctctactcgagtagagcagacagatagcacctttttt

SEQ ID NO: 2 - RNA, 57 nt, synthetic construct
  source 1..57: mol_type = other RNA
ccgggcttggaatgagactgctgatctcgagatcagcagtctcattccaagcttttt

SEQ ID NO: 3 - DNA, 22 bp, synthetic construct
  source 1..22: mol_type = other DNA
aagctccttctggacgaatgtc

SEQ ID NO: 4 - DNA, 22 bp, synthetic construct
  source 1..22: mol_type = other DNA
aatccatccagcccatgtagtc

SEQ ID NO: 5 - DNA, 22 bp, Synthetic construct
  source 1..22: mol_type = other DNA
tcatctccaagctgtcacactc

SEQ ID NO: 6 - DNA, 22 bp, Synthetic construct
  source 1..22: mol_type = other DNA
gttctcaaactcacacccttgc

SEQ ID NO: 7 - DNA, 22 bp, Synthetic construct
  source 1..22: mol_type = other DNA
ttgagaagaacgggaaggtcag

SEQ ID NO: 8 - DNA, 22 bp, Synthetic construct
  source 1..22: mol_type = other DNA
gtttccccttcttgttcatggc

SEQ ID NO: 9 - DNA, 22 bp, Synthetic construct
  source 1..22: mol_type = other DNA
caacgaccactttgtcaagctc

SEQ ID NO: 10 - DNA, 22 bp, Synthetic construct
  source 1..22: mol_type = other DNA
tctcttcctcttgtgctcttgc

SEQ ID NO: 11 - DNA, 22 bp, Synthetic construct
  source 1..22: mol_type = other DNA
tcaaggctgaactcaagaaggc

SEQ ID NO: 12 - DNA, 22 bp, Synthetic construct
  source 1..22: mol_type = other DNA
ggtgcgtcctttaatcctcttc

SEQ ID NO: 13 - DNA, 20 bp, Synthetic construct
  source 1..20: mol_type = other DNA
ggtgtccatcaacaacatgg

SEQ ID NO: 14 - DNA, 18 bp, Synthetic construct
  source 1..18: mol_type = other DNA
gccttgaagagcgcgtag

SEQ ID NO: 15 - DNA, 22 bp, Synthetic construct
  source 1..22: mol_type = other DNA
gtgctgaaggctgattacgttc

SEQ ID NO: 16 - DNA, 22 bp, Synthetic construct
  source 1..22: mol_type = other DNA
tgtagtccaagttgccagtcac

SEQ ID NO: 17 - DNA, 22 bp, Synthetic construct
  source 1..22: mol_type = other DNA
tctgagggtttctccggatttg

SEQ ID NO: 18 - DNA, 22 bp, Synthetic construct
  source 1..22: mol_type = other DNA
gcatttgatcccgtacaacctg

SEQ ID NO: 19 - DNA, 22 bp, Synthetic construct
  source 1..22: mol_type = other DNA
gtcccctggattttgcattcac

SEQ ID NO: 20 - DNA, 22 bp, Synthetic construct
  source 1..22: mol_type = other DNA
ataatcgccgccacaaactctc

SEQ ID NO: 21 - DNA, 22 bp, Synthetic construct
  source 1..22: mol_type = other DNA
tcgatctgccctcctaaaaagc

SEQ ID NO: 22 - DNA, 22 bp, Synthetic construct
  source 1..22: mol_type = other DNA
tcagtaccggaagctgttacag

SEQ ID NO: 23 - DNA, 22 bp, Synthetic construct
  source 1..22: mol_type = other DNA
tcgcctttatggaccacatacg

SEQ ID NO: 24 - DNA, 22 bp, Synthetic construct
  source 1..22: mol_type = other DNA
ttccacatgggctacatcagag

SEQ ID NO: 25 - DNA, 22 bp, Synthetic construct
  source 1..22: mol_type = other DNA
agaagatggtcccagagcaatg

SEQ ID NO: 26 - DNA, 22 bp, Synthetic construct
  source 1..22: mol_type = other DNA
aatctgcttcagaacccaggtc

SEQ ID NO: 27 - DNA, 22 bp, Synthetic construct
  source 1..22: mol_type = other DNA
gtggatgaagcaaatcgtgtcc

SEQ ID NO: 28 - DNA, 22 bp, Synthetic construct
  source 1..22: mol_type = other DNA
tgaatcgtgtctcctcgaacac

SEQ ID NO: 29 - DNA, 22 bp, Synthetic construct
  source 1..22: mol_type = other DNA
tcatcagtaccactctgtgcac

SEQ ID NO: 30 - DNA, 22 bp, Synthetic construct
  source 1..22: mol_type = other DNA
gagtgcagatgtgaacattggg

SEQ ID NO: 31 - DNA, 24 bp, Synthetic construct
  source 1..24: mol_type = other DNA
ttcaccaaagatctgctcctcgct

SEQ ID NO: 32 - DNA, 26 bp, Synthetic construct
  source 1..26: mol_type = other DNA
ttattactggtgtggagtgggtgtgg

SEQ ID NO: 33 - DNA, 22 bp, Synthetic construct
  source 1..22: mol_type = other DNA
acaattcctggcgatacctcag

SEQ ID NO: 34 - DNA, 22 bp, Synthetic construct
  source 1..22: mol_type = other DNA
gttgatgtccacttgcagtgtg

SEQ ID NO: 35 - DNA, 22 bp, Synthetic construct
  source 1..22: mol_type = other DNA
ggtttcagtggtttggatggtg

SEQ ID NO: 36 - DNA, 22 bp, Synthetic construct
  source 1..22: mol_type = other DNA
tctgaccaggagctccattttc

SEQ ID NO: 37 - DNA, 22 bp, Synthetic construct
  source 1..22: mol_type = other DNA
cactggtgatcctggcaaaaac

SEQ ID NO: 38 - DNA, 22 bp, Synthetic construct
  source 1..22: mol_type = other DNA
caccttttccaccttgaacacc

SEQ ID NO: 39 - DNA, 22 bp, Synthetic construct
  source 1..22: mol_type = other DNA
gtcaagggtgaaagtgggaaac

SEQ ID NO: 40 - DNA, 22 bp, Synthetic construct
  source 1..22: mol_type = other DNA
gagccattttcaccacgatcac

SEQ ID NO: 41 - DNA, 22 bp, Synthetic construct
  source 1..22: mol_type = other DNA
caagtggcacagaattgctctc

SEQ ID NO: 42 - DNA, 22 bp, Synthetic construct
  source 1..22: mol_type = other DNA
cacgatgatgccattgatgtcg

SEQ ID NO: 43 - DNA, 20 bp, Synthetic construct
  source 1..20: mol_type = other DNA
tcccctctcgcctatgtctc

SEQ ID NO: 44 - DNA, 22 bp, Synthetic construct
  source 1..22: mol_type = other DNA
gttttgctcacttgcttgcttg